(19) United States
(12) Patent Application Publication
(10) Pub. No.: US 2013/0122082 A1
(43) Pub. Date: May 16, 2013

(54) Methods And Compositions For The Treatment And Prevention Of Aging-Associated Conditions

(71) Applicant: National Yang Ming University, Taipei (TW)

(72) Inventors: Ting-Fen Tsai, Taipei (TW); Yi-Fan Chen, Taipei (TW); Chia-Yu Wu, Taipei (TW); Chih-Hao Wang, Taipei (TW); Shih-Feng Tsai, Taipei (TW)

(73) Assignee: NATIONAL YANG MING UNIVERSITY, Taipei (TW)

(21) Appl. No.: 13/668,268

(22) Filed: Nov. 3, 2012

Related U.S. Application Data
(60) Provisional application No. 61/555,182, filed on Nov. 3, 2011.

(51) Int. Cl. A61K 48/00; A61K 38/17; A61K 45/06; C12N 15/85

(52) U.S. Cl. CPC A61K 48/00; C12N 15/85; A61K 38/17; A61K 45/06
USPC 424/450; 514/44 R; 435/320.1

(57) ABSTRACT

The present disclosure provides novel compositions and therapeutics and their methods of use. In particular, it relates to novel compositions and therapeutics expressing the Cisd2 gene and increasing Cisd2 protein activity as well as methods of using the compositions and therapeutics for treating or preventing aging-associated conditions.

### CROSS-REFERENCE TO RELATED APPLICATION

This application claims the benefit of U.S. Provisional Application No. 61/555,182, filed Nov. 3, 2011, which is incorporated herein by reference in its entirety.

### TECHNICAL FIELD

The present disclosure relates to novel compositions and their methods of use. In particular, it relates to novel compositions and therapeutics comprising the Cisd2 gene and methods of use in treating and preventing aging-associated conditions.

### BACKGROUND

Since the beginnings of time humans have been searching for the secrets of the fountain of youth in order to promote longevity and help maintain the quality of life in the old age. In additional to environmental factors such as nutrition control of diet and caloric restriction that can modulate lifespan, there is substantial evidence to support the familial aggregation of exceptional longevity in humans, which suggests that there is a genetic factor or factors associated with a long life.

In 2001, Perls and Kunkel and their colleagues conducted an open ended search designed to pick up any genetic region that confers exceptional longevity in humans. They carried out a genome-wide scan of long-lived families using 308 individuals who belonged to 137 sets of extremely old siblings; this linkage study identifies a locus on chromosome 4q and suggests that there is a genetic component that contributes significantly to longevity (Puca et al., Proc. Natl. Acad. Sci. USA 98, 10505-10508 (2001)). This genetic component may consist of one or more genes that confer a substantial genetic advantage that leads to survive to extreme old age, defined as outliving by 20-25 years the average life expectancy of a human being. In 2003, Geesaman et al. performed a haplotype-based fine mapping study of the candidate interval on chromosome 4q to identify specific gene and gene variants impacting lifespan using a U.S. cohort of long-lived individuals (mean 100.8 years old); this association study identified a haplotype marker within the microsomal transfer protein (MTP) gene that may function as a modifier of human lifespan (Geesaman et al., Proc. Natl. Acad. Sci. USA 100, 14115-14120 (2003)). To investigate if the result can be replicated in different human populations, several European groups also performed various detailed genetic analyses and a longitudinal studies using European samples of nonagenarians individuals collected in Germany, Denmark, Netherlands and England (Nebel et al., Proc. Natl. Acad. Sci. USA 102, 7906-7909 (2005); Bathum et al., Eur. J. Hum. Genet. 13, 1154-1158 (2005); Beekman et al., Biol. Sci. Med. Sci. 61, 355-362 (2006); Neville et al., Biol. Sci. Med. Sci. 62, 202-205 (2007)). However, these later studies revealed no evidence supporting an association between the MTP gene and longevity and indicated that a noteworthy influence of a MTP haplotype on human longevity is unlikely. Although there are many articles that have examined the process of aging in a variety of experimental organisms, it is extremely valuable to get information about humans. Accordingly, the linkage study of long-lived sibling pairs in humans represents a significant advance (Puca et al., Proc. Natl. Acad. Sci. USA 98, 10505-10508 (2001)), because it points toward an area for further exploration. However, the suspect region on chromosome 4q spans ˜12 Mb and contains hundreds of candidate genes. In this context the winner in this contest would be a new gene encoding a product involved in a new physiological pathway or a new member of a pathway already identified.

The Cisd2 gene, which encodes a mitochondrial outer membrane protein, is an evolutionarily conserved gene (Chen et al., Ann. N.Y. Acad. Sci. 1201, 58-64 (2010)). Cisd2 is the second member of the gene family containing the CDGSH iron sulfur domain. There are currently three members in this gene family: Cisd1 (synonym ZCD1, mitoNEET), Cisd2 (synonym ZCD2, Noxp70, Miner1) and Cisd3 (synonym Miner2). Cisd1 is an outer mitochondria membrane protein that was originally identified as a target protein of the insulin sensitizer drug pioglitazone used to treat type H diabetes. Cisd1 protein contains a transmembrane domain, a CDGSH domain and a conserved amino acid sequence for iron binding; biochemical experiments suggest that Cisd1 is involved in the control of respiratory rates and regulates oxidative capacity. However, Cisd2 and Cisd3 are novel genes with previously uncharacterized functions. The only molecular documentation for Cisd2 is that Cisd2 was one of the markers for early neuronal differentiation in a cell culture study.

Significantly, the Cisd2 gene is located within the candidate region on chromosome 4q where a genetic component for human longevity has been mapped. Previously, a Cisd2-deficient knockout (KO) mouse was engineered in order to study the role of Cisd2 in development and pathophysiology (Chen et al., Genes Dev. 23, 1183-1194 (2009); Autophagy 5, 1043-1045 (2009)). The results clearly revealed that Cisd2 deficiency gives rise to premature aging in mice and demonstrated that Cisd2 is an essential part of mammalian lifespan control. Furthermore, cell culture and submitochondrial fractionation experiments revealed that Cisd2 is a mitochondrial outer membrane protein. Thus, deficiency in the Cisd2 protein leads to mitochondrial degeneration and dysfunction, which is accompanied by cell death that has autophagic features; these events precede neuron and muscle degeneration and, together, these changes lead to a panel of phenotypic features suggestive of premature aging. Since muscles and nerves have the highest energy needs and are therefore most dependent on mitochondrial function, this provides an explanation as to why neuronal lesions and muscle abnormalities are the two earliest manifestations and why they precede the gross premature aging phenotype. Accordingly, mitochondrial degeneration appears to have a direct phenotypic consequence that triggers the accelerated aging process in Cisd2 KO mice. The results thus provided strong evidence for the causal involvement of mitochondrial dysfunction in driving mammalian aging.

Many genetic factors have the potential to modulate lifespan (Kuro-o et al. Nature 390, 45-51 (1997); Hasty et al. Science 299, 1355-1359 (2003); Mounkes et al. Nature 423, 298-310 (2003); Niedernhofer et al. Nature 444, 1038-1043 (2006); Chen et al., Ageing Res. Rev. 9S, S28-S35 (2010)). However, thus far, Cisd2 is the only identified gene that resides in the longevity region of 4q and has been demonstrated to be an essential gene for lifespan control by a loss-of-function mouse study. Nonetheless, experiments that shorten lifespan are likely to be less informative than those that prolong a healthy lifespan. Accordingly, it is important to evaluate the life history of transgenic mice expressing an elevated level of the Cisd2 protein in order to see whether increased Cisd2 promotes longevity.

### SUMMARY

The present disclosure provides novel compositions and their methods of use. More particularly, the inventors have identified novel compositions and therapeutics comprising the Cisd2 gene and methods of use in treating and/or preventing aging-associated conditions.

Accordingly, the present disclosure provides a therapeutic for treating and/or preventing aging-associated damage comprising a delivery vehicle carrying a Cisd2 gene.

In some embodiments of the present disclosure, the Cisd2 gene is selected from the group consisting of human Cisd2 gene and murine Cisd2 gene.

In some embodiments of the present disclosure, the delivery vehicle is a vector, a liposome, a polymer, a pharmaceutically acceptable composition, or a device which facilitates delivery of such delivery vehicle.

In some embodiments of the present disclosure, the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments of the present disclosure, the therapeutic is useful in treating aging-associated damage. In other embodiments of the present disclosure, the therapeutic is useful in preventing aging-associated damage.

In some embodiments of the present disclosure, the aging-associated damage is selected from the group consisting of cell injury, tissue damage, organ dysfunction, aging-associated lifespan shortening and carcinogenesis.

In some embodiments of the present disclosure, the aging-associated damage is associated with a tissue selected from the group consisting of skin, neuron, muscle, pancreas, brain, kidney, lung, stomach, intestine, spleen, heart, adipose tissue, testes, ovary, uterus, liver and bone. The skin may comprise epidermis, dermis, adipose layers, hair follicles, hair shafts, and sebaceous glands.

In some embodiments of the present disclosure, the delivery vehicle is combined with another unrelated therapy.

The present disclosure also provides an expression cassette comprising a polynucleotide comprising a sequence encoding a Cisd2 polypeptide, operably linked to a promoter functional in a host cell.

In some embodiments of the present disclosure, the expression cassette comprises a Cisd2 polypeptide selected from the group consisting of human Cisd2 polypeptide and murine Cisd2 polypeptide.

In some embodiments of the present disclosure, the expression cassette comprises a promoter that is a tissue specific promoter.

The present disclosure further provides a method of treating and/or preventing at least one aging-associated condition comprising administering to a mammal in need of such treatment a therapeutically effective amount of a Cisd2 gene.

In some embodiments of the present disclosure, the method comprises a Cisd2 gene selected from the group consisting of human Cisd2 gene and murine Cisd2 gene.

In some embodiments of the present disclosure, the method comprises an administering step using a delivery vehicle.

In some embodiments of the present disclosure, the method comprises a delivery vehicle selected from the group of a vector, a liposome, a polymer, a pharmaceutically acceptable composition, or a device which facilitates delivery of such delivery vehicle.

In some embodiments of the present disclosure, the method comprises a vector selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments of the present disclosure, the method comprises an administering step which includes administering in a manner selected from the group consisting of intravenous administration intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intra-rectal administration, intra-tracheal injection, intra-nasal administration, intradermal administration, epidermal administration, oral administration and combinations thereof.

In some embodiments of the present disclosure, the method comprises treating and/or preventing aging-associated condition selected from the group consisting of cell injury, tissue damage, organ dysfunction, aging-associated lifespan shortening and carcinogenesis.

In some embodiments of the present disclosure, the method comprises an aging-associated condition that is associated with a tissue selected from the group consisting of skin, neuron, muscle, pancreas, brain, kidney, lung, stomach, intestine, spleen, heart, adipose tissue, testes, ovary, uterus, liver and bone. The skin may comprise epidermis, dermis, adipose layers, haft follicles, haft shaft, and sebaceous glands

In some embodiments of the present disclosure, the method comprises an administering step that includes administering to the mammal in need of treatment multiple therapeutically effective amounts of the Cisd2 gene.

In some embodiments of the present disclosure, the method comprises an administering step that includes administering the Cisd2 gene in combination with another therapeutic.

In some embodiments of the present disclosure, the method comprises treating and/or preventing at least one aging-associated condition in a mammal that is a human.

The present disclosure also provides a transgenic mouse expressing mouse Cisd2. In some embodiments, the present disclosure provides a transgenic mouse whose genome comprises a transgene encoding a mouse Cisd2, wherein expression of the transgene results in over expression of mouse Cisd2, as compared to a non-transgenic mouse. In some embodiments, the present disclosure provides a transgenic mouse whose somatic and germ cells comprise a transgene encoding mouse Cisd2, wherein the expression of the transgene results in over expression of the mouse Cisd2 in all cells, as compared to non-transgenic cells. In some embodiments, the present disclosure provides a transgenic mouse whose genome comprises a transgene encoding a mouse Cisd2, wherein the transgenic mouse exhibits a characteristic selected from the group consisting of decreased skin degeneration, decreased skeletal muscle degeneration, decreased neuronal degeneration, decreased mitochondrial damage, increased metabolism and increased lifespan.

In some embodiments of the present disclosure, the transgenic mouse comprises a transgene that is operatively linked to a promoter that can increase the Cisd2 expression in the transgenic mouse. The promoter may be an RNA polymerase II large subunit (Pol II) promoter.

The present disclosure also provides a method for generating a transgenic mouse comprising the step of introducing a transgene encoding mouse Cisd2 into an ES cell or a germ cell.

The present disclosure also provides a progeny of the transgenic mouse, wherein the progeny expresses a transgene encoding mouse Cisd2.

The present disclosure also provides a cell isolated or derived from the transgenic mouse. In some embodiments, the present disclosure provides a cell obtained or derived from the transgenic mouse, wherein said cell expresses a transgene encoding mouse Cisd2.

The present disclosure further provides a method of screening for compounds capable of modulating Cisd2 expression comprising the steps of: (a) providing a test compound; (b) contacting a transgenic mouse or a cell obtained or derived from the transgenic mouse of with the test compound; and (c) detecting whether the compound is capable of modulating Cisd2 expression.

The present disclosure also provides a method of identifying an agent that modulates a characteristic of an age-associated condition, the method comprising: (a) providing a transgenic mouse whose genome comprises a transgene encoding a mouse Cisd2, and which, compared with non-transgenic mice, exhibits a characteristic selected from the group consisting of decreased skin degeneration, decreased skeletal muscle degeneration, decreased neuronal degeneration, decreased mitochondrial damage, increased metabolism and increased lifespan; (b) measuring said characteristic of the transgenic mouse; (c) comparing the measured characteristic of (b) with that of a non-transgenic mouse; (d) administering a test agent to the transgenic mouse of (a); and (e) determining whether the test agent modulates the characteristic of an age-associated condition.

These and other features, aspects and advantages of the present disclosure will become better understood with reference the following description, examples and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Persistent Expression of Cisd2 Promotes Longevity in Mice. (A) The Pol II-Cisd2 transgenic construct. The mouse Cisd2 coding region is driven by the RNA polymerase II large subunit promoter (Pol II pro.); two direct repeats of the chicken HS4 insulator were placed downstream of the polyA (pA) signal to block positional effects. The Cisd2 TG mice were generated with a C57BL/6 background. (B) Northern blot analysis of the endogenous (endo) and transgenic (tg) Cisd2 mRNA of Cisd2 TG mouse at 3-mo using mouse Cisd2 cDNA as the probe. (C) Quantification of Cisd2 protein levels in the skeletal muscles by Western blot analyses for WT and Cisd2 TG mice at 3-mo, 12-mo and 24-mo. *p<0.05; **p<0.005. (D) (E) Dose-dependent modulation of lifespan by Cisd2 in male and female mice. Cisd2 deficiency shortens the lifespan and causes premature aging in both the male and female Cisd2 KO mice. In contrast, an elevated level of Cisd2 prolongs lifespan and increases the survival rate of both male and female Cisd2 TG mice.

FIG. 2. Effects of Cisd2 on Fur Appearance, Sebaceous Glands and Water Repulsion. (A) Delayed de-pigmentation in the Cisd2 TG mice. There are less grey hairs present in the 34-mo Cisd2 TG mouse compared with a 15-mo WT mouse. (B) (C) Masson's trichome staining of skin section for WT and Cisd2 TG mice at 3-mo. HF, hair follicles; SG, sebaceous gland. Connective tissues, principally collagen, are stained blue by Masson's trichome staining. (D) (E) Masson's trichome staining of skin sections from WT and Cisd2 TG mice at 24-mo. (F) Quantification of nuclear numbers in sebaceous gland (SG) of single hairs. (G) Percentage of hairs with one SG or two SG per individual hair. In (F) and (G), there were 3-6 mice in each group; for each mouse, 40-180 hairs were examined. The results are shown as the mean±SD. *p<0.05. (H) Improved water repulsion in Cisd2 TG mice at 24-mo. The WT mice retained more water in their fur than did the Cisd2 TG mice, which is reflected as a significant increase in relative body weight.

FIG. 3. Cisd2 Protects Mitochondria from Age-Associated Structural Damage, Delays Skeletal Muscle Degeneration, and Increases Insulin Sensitivity. (A) (B) H&E staining of transverse sections of the skeletal muscles from 3-mo WT and Cisd2 TG mice. No difference was detected at this young age. (C) (D) H&E staining of transverse sections of the skeletal muscles from 24-mo WT and Cisd2 TG mice. Stars indicate white adipose tissue substituting for muscle fibers. Degenerated fibers and white adipose were not detected in the Cisd2 TG mice at 24-mo. (E) White adipose tissue, which substitutes for the muscle fibers in the skeletal muscle of aged WT mice, was confirmed by IHC staining for perilipin. Perilipin is a marker for adipose that is expressed at the periphery of lipid droplets. The blue color is nuclear staining by DAPI. (F) H&E staining of a serial section of the skeletal muscle examined in panel E. (G) Significant increase in the area occupied by muscle fibers in the skeletal muscle of Cisd2 TG mice at 24-mo. (H) Significant increase in the fiber numbers in the skeletal muscle of Cisd2 TG mice at 24-mo. (I) Significant decrease in the area of white adipose tissue substitution in the skeletal muscle of Cisd2 TG mice at 24-mo. In (G)-(I), there were 3-4 mice in each group; 3-5 microphotographs (400×) for each mouse were examined. (J) Functional examination of muscle strength using grip strength analysis. *p<0.05. (K) (L) TEM examination of the ultrastructure of skeletal muscles for WT and Cisd2 TG mice at 24-mo. AL, Autolysosomes; M, mitochondria; Myf, myofilament. (M) Percentage of the skeletal muscle showing degenerative areas and autophagic vacuoles at 24-mo. (N) Percentage of area occupied by mitochondria (mt) in the skeletal muscle at 24-mo. In (M) and (N), there were 3 mice in each group; 10-12 microphotographs (5,000×) for each mouse were examined. (O) Increased insulin sensitivity of the Cisd2 TG mice at 12-mo and 24-mo. Insulin (0.75 U/kg body weight) tolerance tests (ITT) were performed at 3-mo, 12-mo and 24-mo. There were 3-6 mice in each group; three independent measurements were carried out for each mouse. *p<0.05.

FIG. 4. Cisd2 Protects Mice from Age-Associated Neuron Degeneration and Myelin Sheath Disintegration. (A) (B) Ultrastructure of the sciatic nerve of WT mice at 24-mo. Disintegration of the myelin sheath and degenerating axonal components are evident. (C) (D) Ultrastructure of the optic nerve of WT mice at 24-mo. Disintegration of the myelin sheath and degenerating axonal components are evident. (E) (F) Ultrastructure of the sciatic nerve of the Cisd2 TG mice at 24-mo. (G) (H) Ultrastructure of the optic nerve of the Cisd2 TG mice at 24-mo. (I) (J) Quantification of the TEM examination for the sciatic nerve at 24-mo. There were 3 mice in each group; 3-5 microphotographs (5,000×) for each mouse were examined. (K) (L) Quantification of the TEM examination for the optic nerve at 24-mo. There were 3 mice in each group; 10 microphotographs (10,000×) for each mouse were examined. The results are shown as the mean±SD. *p<0.05; **p<0.005.

FIG. 5. Cisd2 Reduces Age-Associated Mitochondrial DNA Damages and Attenuates Age-Associated Decline in the Electron Transport Activities of Mitochondria. (A) Map of the mtDNA genome and the positions of primer pairs used to detect deletions. ND1, NADH dehydrogenase subunit 1; COXI, cytochrome c oxidase subunit I; ATP6, ATP synthase F0 subunit 6; CYTB, cytochrome b. (B) Long PCR (13.6-kb) of mtDNA fragment, which was used to monitor the integrity of the mitochondrial genome using genomic DNA isolated from liver. (C) Comparison of the relative percentage of 13.6-kb long PCR product and deleted mtDNA PCR signals between WT and Cisd2 TG mice at 24-mo. (D) PCR detection of the D-17 deletion, which covers the ND1 and ND2 genes using genomic DNA isolated from liver. Manba is a nuclear-encoded protein. (E) Comparison of the relative levels of D-17 deletion between WT and Cisd2 TG mice at 24-mo. (F) Quantification of the protein levels of NDUFA, UQCRC2 and ATP5B analyzed by Western blot. (G) The mitochondrial content (weight) was significantly decreased in the skeletal muscle of WT mice at 24-mo; however, this decrease was attenuated in the Cisd2 TG mice. (H) mtDNA copy number was significantly increased in the skeletal muscle of WT mice at 24-mo; however, this increase was not detected in the Cisd2 TG mice. (I) TFAM was significantly increased in the skeletal muscle of WT mice at 24-mo; however, this phenomenon was not obvious in the Cisd2 TG mice. (J) Respiratory activity of isolated mitochondria expressed as oxygen consumption rate (nanomoles of O2 per minute per gram of skeletal muscle) in the resting state for glutamate-malate supported respiration and for ADP activated respiration. *p<0.05; **p<0.005.

FIG. 6. Cisd2 Reduces the Age-Associated Decline in Whole-Body Energy Metabolism. (A-C) Hour-to-hour average of whole-body oxygen consumption (VO₂) (A), CO2 production (VCO₂) (B), and heat generation (C) during light/dark period for 24-mo WT and Cisd2 TG mice. (D-F) Quantification of whole10 body VO₂ (D), VCO₂ (E), and heat generation (F) during the light and dark periods, and comparison between WT and Cisd2 TG mice at 3-mo and 24-mo. In (D-F), the quantification results were calculated using data collected during the middle of the light period (11:00-13:00) and during dark period (23:00-1:00). All results are mean±SD. *p<0.05; **p<0.005.

FIG. 7. Expression of the Cisd2 mRNA in the Cisd2 TG Mice at Young Age (3-mo) and Middle Age (17-mo) (A) Allele-specific RT-PCR was applied to detect the mRNA expressed from the endogenous (endo) and transgenic (tg) Cisd2 gene. (B) RT-PCR of Cisd2 mRNA using total RNA isolated from different tissues of the transgenic male mice at 3-mo and 17-mo. BAT, brown adipose tissue; WAT, white adipose tissue; Sk. muscle, skeletal muscle.

FIG. 8. Transgenic Expression of Cisd2 Gene Helps Maintain the Protein Level of Cisd2 in the TG Mice; the Expression of the Endogenous Cisd2 is Decreased in An Age-Dependent Manner in Naturally Aged WT Mice. (A) Western blot analyses for detection of the Cisd2 protein level in skeletal muscles of naturally aged WT mice and Cisd2 TG mice at 3-mo, 12-mo and 24-mo. An international control (IC) isolated from a 3-mo skeletal muscle was loaded to each protein gel for comparison and calibrating signals between different western blots. (B) Comparison of the relative levels of Cisd2 protein in the skeletal muscles between WT and Cisd2 TG mice at 3-mo, 12-mo and 24-mo. A significant decrease in the levels of Cisd2 protein was detected in the naturally aged WT mice at 12-mo and 24-mo. However, there is a significantly different expression level of Cisd2 protein from a young age (3-mo) to an old age (24-mo) in the Cisd2 TG mice.

FIG. 9. Comparison of Female Reproduction, Body Weight and Body Temperature between Age- and Sex-Matched WT and Cisd2 TG Mice. (A) No obvious difference in the female fertility between WT and Cisd2 TG mice. The numbers for litters and pups were recorded from 2-mo to 12-mo of the females. The body weight was measured at 6-mo, 12-mo and 24-mo. (C) There is no significant difference in the body temperature when compared between the WT and Cisd2 TG mice at 12-mo. Core temperature of the body was measured by Microcomputer Thermometer MODEL 7000H (JENCO ELECTRONICS. LTD.).

FIG. 10. No Significant Difference in the Metabolic Indexes between Age- and Sex-Matched WT and Cisd2 TG Mice at Middle Age (12-mo). (A) Food consumption. (B) Water drinking. (C) Urine generation. (D) Stool generation.

FIG. 11. Effects of Persistent Expression of Cisd2 on Water Repulsion and Body Temperature at Young Age (3-mo) and Old Age (24-mo) in Mice. (A) Improved water repulsion in Cisd2 TG mice compared with WT mice at 24-mo. WT mice retained more water in their fur than did Cisd2 TG mice, as reflected by a significant increase in relative body weight. No significant difference in water repulsion at 3-mo. There are four mice (n=4) for each group. *p<0.05. (B) No significant difference in body temperature after water immersion between the WT and Cisd2 TG mice at 3-mo and 24-mo.

FIG. 12. Histopathological Analysis Revealed No Significant Difference in the Thickness of Skin Layers between WT and Cisd2 TG Mice at Old Age (24-mo). (A) Representative microphotograph for H&E staining of the skin section of WT mice at 3-mo. (C) Representative microphotograph for H&E staining of the skin section of Cisd2 TG mice at 3-mo. (B) Representative microphotograph for H&E staining of the skin section of WT mice at 24-mo. (D) Representative microphotograph for H&E staining of the skin section of Cisd2 TG mice at 24-mo. (E) Quantification of the thickness of skin, including dermis, adipose and muscle. (F) Quantification of the thickness of dermis layer. (G) Quantification of the thickness of subcutaneous adipose layer. (H) Quantification of the thickness of muscle layer. The SPOT Imaging Software Advance (DIAGNOSTIC Instruments. Inc.) was applied for quantification analysis. In (E)-(H), there were 3 mice for WT group and 4 mice for TG group; 3-9 microphotographs (400×) for each mouse were examined.

FIG. 13. Increased Hair Re-Growth Rate at Middle Life Stage (12-mo) in the Cisd2 TG Mice Compared with Age- and Sex-Matched WT Mice. Representative photographs showing 12-month old Cisd2 TG male mice and age-matched WT male mice 3, 6, 9, 12, 15, 18, 21 days after removal of hair on dorsal area.

FIG. 14. Cisd2 Protects Skeletal Muscles from Age-Associated Degeneration in the Cisd2 TG Mice at Middle Age (15-mo) and Old Age (24-mo). (A) (B) H&E staining of transverse sections of skeletal muscles for WT and Cisd2 TG mice at 3-mo, respectively. No difference was detected at this young age. (C) (D) H&E staining of transverse sections of skeletal muscles for WT and Cisd2 TG mice at 15-mo, respectively. Arrows indicate degenerated regions of the fibers at 15-mo; this fiber degeneration is mainly observed in the WT mice, but not in the Cisd2 TG mice. (E) (F) H&E staining of transverse sections of skeletal muscles for WT and Cisd2 TG mice at 24-mo, respectively, with an original magnification 100×. (G) (H) H&E staining of transverse sections of skeletal muscles for WT and Cisd2 TG mice at 24-mo, respectively, with an original magnification 400×. Stars indicate white adipose tissues substitute for the muscle fibers. Degenerated fibers and white adipose were not detected in the Cisd2 TG mice at 15-mo and 24-mo.

FIG. 15. Cisd2 Protects Mitochondria of the Skeletal Muscle from Age-Associated Degeneration in the Cisd2 TG Mice. (A-C) TEM examination of ultrastructures of skeletal muscles for WT mice at 24-mo with an original magnification of 5,000×(A), 10,000×(B) and 30,000×(C). Arrows indicate autophagic vacuoles which are accompanied by degenerated mitochondria. (D-F) TEM examination of ultrastructures of skeletal muscle for Cisd2 TG mice at 24-mo with an original magnification of 5,000×(D), 10,000× (E) and 30,000× (F). AL, Autolysosomes; M, mitochondria; Myf, myofilament.

FIG. 16. Cisd2 Helps Maintain the Fiber Distribution and Ratios of Type I (Slow-Twitch) to Type II (Fast-Twitch) Fibers during Aging. (A) Immunofluorescence staining of type I and type II fibers of the skeletal muscles for age- and sex-matched WT and Cisd2 TG mice. There was an apparent grouping of both types of fibers observed in the old (24-mo) WT mice; both fiber types seems to be distributed in clusters rather than in a random fashion commonly observed in young (3-mo) muscle. (B) Quantification of type I and type II fibers of skeletal muscle for WT and Cisd2 TG mice at 3-mo and 24-mo. There was a selective amplification of type I fibers, whereas atrophy of type II fibers in the old (24-mo) WT mice; however, increasing age did not change the fiber distribution and ratios of type I to type II in Cisd2 TG mice. There were three mice for each group; 3-5 microphotographs for each mouse were examined. The results were shown as the mean±SD. *p<0.05; **p<0.005.

FIG. 17. No Significant Difference in the Oral Glucose Tolerance Tests (GTT) between Age- and Sex-Matched WT and Cisd2 TG mice at Young Age (3-mo), Middle Age (12-mo) and Old Age (24-mo). (A) GTT for 3-mo WT and Cisd2 TG mice. (B) GTT for 12-mo WT and Cisd2 TG mice. (C) GTT for 24-mo WT and Cisd2 TG mice. Animal numbers are indicated for each group of mice. Three independent measurements were carried out for each mouse.

FIG. 18. The Cisd2 TG Mice Had A Trend toward Better Motor Function in the Test of Open Field Locomotion Compared with Age- and Sex-Matched WT Mice at Middle Age (12-mo) and Old Age (24-mo). (A) Total travelling distance monitored by the sensor ring in floor plane was recorded during a period of 60 minutes. (B) Travelled distance in the center zone of the open-field chamber. (C) The frequency of rearing monitored by the sensor ring in vertical plane was calculated to indicate the exploratory activities of mice. *p<0.05 was considered statistically significant.

FIG. 19. The Cisd2 TG Mice Had A Trend toward Better Motor Function in the Test of Rotarod Trials Compared with Age- and Sex-Matched WT Mice at Old Age (24-mo). (A) Pre-training (3 days) of rotarod for the WT and Cisd2 TG mice at 3-mo and 24-mo. (B) Three conditions of trials (T1, T2, T3) were performed for WT and Cisd2 TG mice at 3-mo and 24-mo. *p<0.05 was considered statistically significant.

FIG. 20. Comparison of D-loop Oxidative Damage, Nuclear-Encoded Mitochondrial Proteins and mtDNA Copy Numbers in the Skeletal Muscles between Age- and Sex-Matched WT and Cisd2 TG Mice. (A) No significant difference for oxidative damage of the D-loop between WT and Cisd2 TG mice at 3-mo, 15-mo and 24-mo. (B) Quantification of protein levels of NDUFA9 (a component of complex I). There was a significant increase of NDUFA9 in Cisd2 TG mice compared with WT mice at 24-mo. (C) Quantification of protein levels of SDHB (a component of complex II). There was no significant difference between WT and Cisd2 TG mice at 3-mo, 12-mo and 24-mo. (D) Quantification of protein levels of UQCRC2 (a component of complex III). In WT mice, there was an age-dependent decrease of UQCRC2 protein level. In addition, there was a significant increase of UQCRC2 in Cisd2 TG mice compared with WT mice at 24-mo. (E) Quantification of protein levels of ATP5B (a component of complex V). There was a significant increase of ATP5B in Cisd2 TG mice compared with WT mice at 24-mo. In (B)-(E), there were 3 mice in each group; relative protein levels were analyzed by western blot using specific antibodies. The results were shown as the mean±SD. *p<0.05; **p<0.005. (F) Age-associated amplification of mtDNA copy number was not observed in Cisd2 TG mice at 24-mo. There was a significant increase of mtDNA copy number in WT mice at 24-mo; this could be attributable to a compensatory amplification of the mtDNA induced by mitochondrial damages in the skeletal muscles of aged WT mice. Detailed methods for D-loop oxidative damage, measurement of mtDNA copy number and western blot analysis of protein levels are described herein.

FIG. 21. Comparison of Electron Transport Activities of the Mitochondrial Respiratory Enzyme Complexes Prepared from the Skeletal Muscles of Age- and Sex-Matched WT and Cisd2 TG Mice. (A) Measurement of NADH dehydrogenase activity, which represents complex I. (B) Measurement of NCCR activity, which represents complexes I to III. (C) Measurement of SCCR activity, which represents complexes II to III. (D) CCO activity, which represents complex IV. In (A)-(D), there were more than four mice of each group. Detailed methods for measurement of respiratory enzyme complex activity are described herein. *p<0.05.

FIG. 22. No Significant Difference in Whole-Body Energy Metabolism between Age- and Sex-Matched WT and Cisd2 TG Mice at Young Age (3-mo). (A) Hour-to-hour average of whole-body O2 consumption (VO2) during light/dark period for 3-mo mice. (B) Hour-to-hour average of whole-body CO2 production (VCO2) during light/dark period for 3-mo mice. (C) Hour-to-hour average of whole-body heat generation during light/dark period for 3-mo mice. (D) Quantification of whole-body VO2 at light and dark periods for WT and Cisd2 TG mice at 3-mo. (E) Quantification of whole-body VCO2 at light and dark periods for WT and Cisd2 TG mice at 3-mo. (F) Quantification of whole-body heat generation at light and dark periods for WT and Cisd2 TG mice at 3-mo. In (D)-(F), the quantification results were calculated using data collected at the middle of light period (11:00-13:00) and dark period (23:00-1:00). All results are mean±SD. *p<0.05; **p<0.005.

FIG. 23. No Significant Difference in Intracellular Glutathione (GSH) and the mRNA Levels of the Enzymes That Scavenge Reactive Oxygen Species (ROS) between WT and Cisd2 TG Mice at Young Age (3-mo) and Old (24-mo) Age. (A) Quantification of intracellular GSH levels in the skeletal muscles. (B) Quantification of the mRNA levels of catalase, SOD1 (CuZn-SOD) and SOD2 (Mn-SOD) in the skeletal muscles using real-time quantitative RT-PCR. All results are mean±SD. Animal numbers (n) for each group of mice are indicated.

FIG. 24. Survival Rates for Two Independent Lines (A161 and A214) of the Cisd2 TG Mice. Line A214 was created two years after the establishment of line A161; both lines are in C57BL/6 background. We have been preparing adequate numbers of male and female mice to replicate the anti-aging phenotype in line A214. The survival rate for line A214 has been recording for 22 months (as of Jul. 22, 2011). (A) Survival rate for A214 male mice (blue squares). The survival rate of A214 males overlaps with that of A161 males and separates apart from WT males at around 18 months. (B) At 22 months, only 85% of WT males are survived; whereas in Cisd2 TG mice, there is a 93,3% and 100% survival rate for A161 and A214 males, respectively. (C) Survival rate for A214 female mice (red circles). The survival rate of A214 females overlaps with that of A161 females and separates apart from WT females at around 20 months. (D) At 22 months, only 87.5% of WT females are survived; whereas 100% of Cisd2 TG females in both lines are alive.

FIG. 25. Cisd2 Protects Skeletal Muscles from Age-Associated Degeneration in Two Independent Lines (A161 and A214) of the Cisd2 TG Mice. (A) (B) H&E staining of transverse sections of skeletal muscles for WT mice at 24-mo with an original magnification of 100× and 400×, respectively. (C) (D) H&E staining of transverse sections of skeletal muscles for line A161 of the Cisd2 TG mice at 24-mo with an original magnification of 100× and 400×, respectively. (E) (F) H&E staining of transverse sections of skeletal muscles for line A214 of the Cisd2 TG mice at 24-mo with an original magnification of 100×, respectively. Stars indicate white adipose tissues substitute for the muscle fibers. Degenerated fibers and white adipose were not observed in both lines (A161 and A214) of the Cisd2 TG mice at 24-mo.

FIG. 26. Cisd2 Protects Mitochondria of the Skeletal Muscle from Age-Associated Degeneration in Two Independent Lines (A161 and A214) of the Cisd2 TG Mice. (A) (B) TEM examination of ultrastructures of skeletal muscles for WT mice at 24-mo with an original magnification of 10,000× and 30,000×, respectively. Arrows indicate autophagic vacuoles which are accompanied by degenerated mitochondria. (C) (D) TEM examination of ultrastructures of skeletal muscles for line A161 of the Cisd2 TG mice at 24-mo with an original magnification of 10,000× and 30,000×, respectively. (E) (F) TEM examination of ultrastructures of skeletal muscles for line A214 of the Cisd2 TG mice at 24-mo with an original magnification of 10,000× and 30,000×, respectively. AL, Autolysosomes; M, mitochondria; Myf, myofilament.

FIG. 27. Cisd2 Protects Mice from Age-Associated Neuron Degeneration and Myelin Sheath Disintegration. This neuroprotective effect of Cisd2 was observed in two independent lines (A161 and A214) of the Cisd2 TG mice. (A) (B) Ultrastructure of sciatic nerve of WT mice at 24-mo. Disintegrating myelin sheath and degenerating axonal component were evident. (C) (D) Ultrastructure of optic nerve of WT mice at 24-mo. Disintegrating myelin sheath and degenerating axonal component were also evident. (E) (F) Ultrastructure of sciatic nerve of line A161 of the Cisd2 TG mice at 24-mo. (G) (H) Ultrastructure of optic nerve of line A161 of the Cisd2 TG mice at 24-mo. (I) (J) Ultrastructure of sciatic nerve of line A214 of the Cisd2 TG mice at 24-mo. (K) (L) Ultrastructure of optic nerve of line 5 A214 of the Cisd2 TG mice at 24-mo.

FIG. 28. Multiple Sequence Alignment and Phylogenetic Analysis of Cisd2 Homologs. (A) Multiple sequence alignment of Cisd2 homologs which are evolutionarily conserved from mammals to insects. Yellow color indicates that the residues in that column are identical in all sequences in the alignment. Purple indicates that conserved substitutions have been observed. Green indicates that similar substitutions are observed. Multiple sequence alignment of Cisd2 homologs from the 29 species was conducted by the software AlignX of Vector NTI Suite 8. (B) Phylogenetic Analysis: UPGMA Tree (Mega 5) was constructed from the Multiple Alignment (ClustalW) of the Cisd2 amino acids from 29 species. The scale bar represents 10% sequence divergence.

### DETAILED DESCRIPTION OF PREFERRED EMBODIMENTS

Unless otherwise defined, all technical terms used herein have the same meaning as commonly understood by one of ordinary skill in the art to which the present disclosure belongs.

To facilitate understanding of the present application and for ease of reference, a number of terms and abbreviations as used herein are defined below.

As used herein, the terms “treating” and “treatment” are used to refer to administration or application of a therapeutic agent to a subject or performance of a procedure or modality on a subject for the purpose of obtaining a therapeutic benefit of a disease or health-related condition.

As used herein, the terms “preventing,” “inhibiting,” “reducing” or any variation of these terms, includes any measurable decrease or complete inhibition to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99% or more, or any range derivable therein, reduction of activity or symptoms, compared to normal.

As used herein, the terms “administered” and “delivered” are used to describe the process by which a composition of the present disclosure is administered or delivered to a subject, a target cell or are placed in direct juxtaposition with the target cell. The terms “administered” and “delivered” are used interchangeably.

As used herein, the terms “patient,” “subject” and “individual” are used interchangeably herein, and mean a mammalian (e.g., human) subject to be treated and/or to obtain a biological sample from.

As used herein, the term “effective” means adequate to accomplish a desired, expected, or intended result. For example, an “effective amount” may be an amount of a compound sufficient to produce a therapeutic benefit.

As used herein, the terms “therapeutically effective” or “therapeutically beneficial” refers to anything that promotes or enhances the well-being of the subject with respect to the medical treatment of a condition. This includes, but is not limited to, a reduction in the onset, frequency, duration, or severity of the signs or symptoms of a disease.

As used herein, the term “therapeutically effective amount” is meant an amount of a composition as described herein effective to yield the desired therapeutic response.

As used herein, the terms “diagnostic,” “diagnose” and “diagnosed” mean identifying the presence or nature of a pathologic condition.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used as described herein.

The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

Although methods and compositions similar or equivalent to those described herein can be used in the practice or testing of the present disclosure, suitable methods and compositions are described below.

It is specifically contemplated that any limitation discussed with respect to one embodiment of the present disclosure may apply to any other embodiment of the present disclosure. Furthermore, any composition of the present disclosure may be used in any method of the present disclosure, and any method of the present disclosure may be used to produce or to utilize any composition of the present disclosure.

The particular embodiments discussed below are illustrative only and not intended to be limiting.

The present disclosure is directed to novel compositions and therapeutics comprising the Cisd2 gene and uses thereof that will be useful in treating and/or preventing aging-associated conditions. The Cisd2 gene encodes an evolutionarily conserved mitochondrial outer membrane protein. Significantly, the Cisd2 gene is located within the candidate region on chromosome 4q where a genetic component for human longevity has been mapped. Previously, it was demonstrated that Cisd2 deficiency shortens lifespan resulting in premature aging in mice. Additionally, an age-dependent decrease in Cisd2 expression has been detected during normal aging.

The present disclosure provides for the first time that a persistent level of Cisd2 achieved by transgenic expression in mice extends the median and maximum lifespan of mice without any apparent deleterious side effects. The present disclosure also provides that Cisd2 ameliorates age-associated degeneration of the skin, skeletal muscles and neurons. Moreover, the present disclosure provides that Cisd2 protects mitochondria from age-associated damage and functional decline as well as attenuating age-associated reduction in whole-body energy metabolism. The results presented herein suggest that Cisd2 is a fundamentally important regulator of lifespan, and provide an experimental basis for exploring the candidacy of CISD2 in human longevity.

Cisd2 Gene

The term “Cisd2” as used herein includes Mus musculus CDGSH iron sulfur domain 2, Homo sapiens CDGSH iron sulphur domain 2, as well as the orthologous genes including Gret, ZCD2, Miner1 Noxp70, AI848398, 1500009M05Rik, 1500026J14Rik, 1500031D15Rik, and B630006A20Rik.

The Cisd2 gene encodes an evolutionarily conserved mitochondrial outer membrane protein which is located within the candidate region of chromosome 4q where a genetic component for human longevity has been mapped.

The present disclosure provides that maintaining a persistent level of Cids2 expression over the different stages of life promotes longevity and ameliorates age-associated phenotypes in mice. The present disclosure provides evidence using a gain-of-function study of TG mice that demonstrate the relationship between an increase in the level of Cisd2 protein during middle and old ages is able to delay aging and extends healthy lifespan.

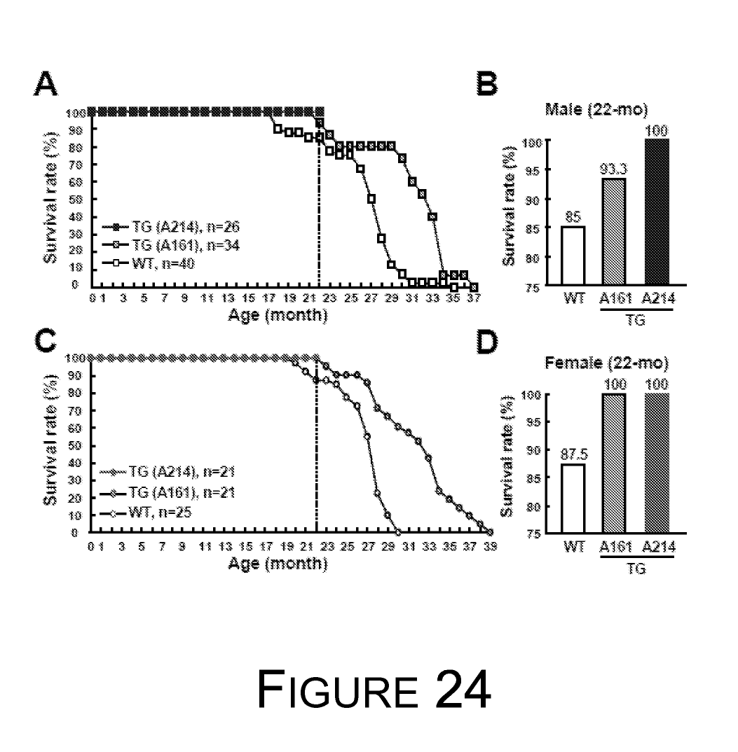

Two independent TG mouse lines were generated, A161 and A214, containing the Pol II-Cisd2 TG construct. Some of the results presented in the Figures and Table described herein were prepared from line A161. Line A214 was created two years after the establishment of line A161 when the line A161 began exhibiting a delay in aging process. This was in order to ascertain whether the anti-aging phenotype mediated by Cisd2 in line A161 could be reproduced in a second independent TG mouse line; thus we also have performed a phenotypic characterization of the line A214. The survival rate of line A214 has been following for 22 months and the survival curves for both males and females of line A214 begun to separate from those of the WT controls and are exhibiting a sign of a longer lifespan (FIG. 24).

As illustrated in Example 2, an enhanced and persistent expression of the Cisd2 gene promotes longevity. The lifespan extension of Cisd2 TG mice (19-20%) was similar in magnitude to that found for MCAT mice (17-21%), which overexpress human catalase targeted to mitochondria, but less than that achieved by caloric restriction (30-50%) or dwarfism (26-68%) or that observed in other genetic models of delayed and decelerated aging. However, the promoting effect of Cisd2 on lifespan is accompanied by no apparent deleterious side effects on growth, fertility, food intake and metabolism. Prolongation of Cisd2 expression into middle and old age seems to have no obvious negative effect that is detrimental to the whole organism.

Regarding lifespan extension in mammals, the most striking prolongation has been reported for the dwarf mice. For example, Ames dwarf mice are remarkably long-lived and outlive their WT controls by 49-68% and exhibit many phenotypic characteristics related to a delay in aging. However, these dwarf mice have a genetic deficit in the growth hormone signaling pathway that leads to a phenotype involving severe growth retardation (dwarfism), sterility or reduced fertility and impaired anterior pituitary development/function. In terms of reproduction, there seems to be generally a reverse correlation between reproduction and lifespan. Most of the long-lived mutant mice show delayed reproductive development and a significantly reduced fertility. In addition to the dwarf mice having severely reduced fertility, the long-lived klotho TG mice, which develop normally and outlived by 19-31% their normal littermates, also display reduced fecundity. In contrast to these earlier findings on extending the lifespan of mice, the TG mouse of the present disclosure provides strong evidence to indicate that persistent expression of Cisd2 is able to extend healthy lifespan without any detectable deficit in development and physiological functioning.

The present disclosure thus provides for a transgenic animal expressing the Cisd2 gene. In some embodiments, the transgenic animal of the present disclosure can be any non-human mammal, preferably a mouse. A transgenic animal can also be, for example, any other non-human mammals, such as rat, rabbit, goat, pig, dog, cow, or a non-human primate. It is understood that transgenic animals that express the Cisd2 gene, as disclosed herein, or other mutated forms that increase or enhance the expression of Cisd2, can be used in methods of the present disclosure.

In some embodiments, the present disclosure provides a transgenic mouse expressing mouse Cisd2. In other embodiments, the present disclosure provides a transgenic mouse whose genome comprises a transgene encoding a mouse Cisd2, wherein expression of the transgene results in over expression of mouse Cisd2, as compared to a non-transgenic mouse. In other embodiments, the present disclosure provides a transgenic mouse whose somatic and germ cells comprise a transgene encoding mouse Cisd2, wherein expression of the transgene results in over expression of the mouse Cisd2 in all cells, as compared to non-transgenic cells. In still other embodiments, the present disclosure provides a transgenic mouse whose genome comprises a transgene encoding a mouse Cisd2, wherein the transgenic mouse exhibits a characteristic selected from the group consisting of decreased skin degeneration, decreased skeletal muscle degeneration, decreased neuronal degeneration, decreased mitochondrial damage, increased metabolism and increased lifespan.

In some embodiments, the transgenic mice of the present disclosure comprise a transgene encoding mouse Cisd2 that is operatively linked to a promoter that can increase the Cisd2 expression in the transgenic mouse. The promoter may be an RNA polymerase II large subunit (Pol II) promoter.

The present disclosure also provides a method for generating a transgenic mouse described herein comprising the step of introducing a transgene encoding mouse Cisd2 into an ES cell or a germ cell.

The present disclosure further provides for a progeny of the transgenic mouse described herein, wherein the progeny expresses a transgene encoding mouse Cisd2.

The present disclosure still further provides for a cell isolated or derived from the transgenic mouse described herein. In some embodiments, the present disclosure provides a cell obtained or derived from the transgenic mouse described herein, wherein said cell expresses a transgene encoding mouse Cisd2. The cell or cell line may be an undifferentiated cell which is selected from the group consisting of a stem cell, embryonic stem cell oocyte and embryonic cell.

Therapeutic Uses

The present disclosure provides that maintenance of a persistent expression level of Cisd2 over the whole life, when achieved by transgenic expression, protects mice from age-associated mitochondrial damages in terms of both genomic DNA and at the ultrastructural level as well as attenuating age-associated functional decline with respect to energy metabolism and oxidative phosphorylation of mitochondria. At the organism level, this protection mediated by Cisd2 contributes to the alleviation of age-associated phenotypic changes in multiple tissues including skin, muscle and neurons. Additionally, Cisd2 helps improve insulin sensitivity and preserve whole-body energy metabolism in the TG mice. Together, these changes give the mice a long-lived phenotype that is linked to an extension in healthy lifespan and a delay in age-associated diseases.

In particular, as illustrated in Example 3, an enhanced and persistent expression of the Cisd2 gene delays skin aging, more specifically, the age-dependent atrophy of the sebaceous glands. Cisd2 expression also delays muscle aging, in particular, age-dependent mass losses and prevents fat infiltration, as illustrated in Example 4. The Cisd2 expression confers protection from age-associated degeneration of mitochondria within the muscle. Example 4 further illustrates improved insulin sensitivity in the presence of persistent Cisd2 expression. As illustrated in Example 5, persistent expression of the Cisd2 gene also delays neuron aging, and in particular, age-associated damage to the nerves. Example 6 further illustrates that persistent expression of the Cisd2 gene protects mitochondria from age-associated damage, reduces age-associated declines in mitochondrial function and whole-body energy metabolism.

The present disclosure thus provides a therapeutic for treating and/or preventing aging-associated damage, wherein the therapeutic comprising a delivery vehicle carrying a Cisd2 gene.

In some embodiments, the therapeutic comprises a Cisd2 gene that is a human Cisd2 gene or a murine Cisd2 gene.

In some embodiments, the delivery vehicle carrying the Cisd2 gene may be a vector, a liposome, a polymer, a pharmaceutically acceptable composition, or a device which facilitates delivery of such delivery vehicle.

In particular, the vector may be selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

The present disclosure provides that the therapeutic may be useful for treating and/or preventing aging-associated damage. The aging-associated damage according to the present disclosure is selected from the group consisting of cell injury, tissue damage, organ dysfunction, aging-associated lifespan shortening and carcinogenesis. The present disclosure also provides that the aging-associated damage is associated with skin, neuron, muscle, pancreas, brain, kidney, lung, stomach, intestine, spleen, heart, adipose tissue, testes, ovary, uterus, liver and bone. The skin may comprise epidermis, dermis, adipose layers, hair follicles, hair shaft, and sebaceous glands.

The present disclosure provides that the Cisd2 gene plays a pivotal role in slowing the aging process by delaying physiological decline and preventing the onset of age-associated diseases. This, in turn, improves overall health and maintains a higher quality of life during old age. The ability to stimulate Cisd2 expression and/or activity to ameliorate age-associated phenotypes such as muscle and neuronal degeneration leads to an extension of a healthy lifespan. Thus, the therapeutics of the present disclosure may be useful in preventing and/or treating aging-associated damage associated with any tissue or organ, including skin, neuron, muscle, pancreas, brain, kidney, lung, stomach, intestine, spleen, heart, adipose tissue, testes. ovary, uterus, liver and bone. The skin may comprise epidermis, dermis, adipose layers, haft follicles, haft shaft, and sebaceous glands.

The present disclosure also provides an expression cassette comprising a polynucleotide comprising a sequence encoding a Cisd2 polypeptide operably linked to a promoter functional in a host cell. The Cisd2 polypeptide can be human Cisd2 polypeptide and murine Cisd2 polypeptide. The promoter may be a tissue specific promoter.

A number of viral based systems have been developed for gene transfer into mammalian cells. For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo.

A number of adenovirus vectors have also been described. Unlike retroviruses which integrate into the host genome, adenoviruses persist extrachromosomally thus minimizing the risks associated with insertional mutagenesis.

Additionally, various adeno-associated virus (AAV) vector systems have been developed for gene delivery. AAV vectors can be readily constructed using techniques well known in the art.

Additional viral vectors which will find use for delivering the nucleic acid molecules encoding the Cisd2 gene include those derived from the pox family of viruses, including vaccinia virus and avian poxyvirus. Alternatively, avipoxyiruses, such as the fowlpox and canarypox viruses, can also be used to deliver the Cisd2 gene.

Molecular conjugate vectors, such as the adenovirus chimeric vectors described in Michael et al., J. Biol. Chem. 268, 6866-6869 (1993) and Wagner et al., Proc. Natl. Acad. Sci. USA 89, 6099-6103 (1992), can also be used for gene delivery.

Members of the Alphavirus genus, such as, but not limited to, vectors derived from the Sindbis, Semliki Forest, and Venezuelan Equine Encephalitis viruses, will also find use as viral vectors for delivering the polynucleotides of the present disclosure.

A vaccinia based infection/transfection system can be conveniently used to provide for inducible, transient expression of the coding sequences of interest in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA which is then translated into protein by the host translational machinery. The method provides for high level, transient, cytoplasmic production of large quantities of RNA and its translation products.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase.

The synthetic expression cassettes of interest can also be delivered without a viral vector. For example, the synthetic expression cassettes can be packaged as DNA or RNA in liposomes prior to delivery to the subject or to cells derived therefrom. Lipid encapsulation is generally accomplished using liposomes which are able to stably bind or entrap and retain nucleic acid. The ratio of condensed DNA to lipid preparation can vary but will generally be around 1:1 (mg DNA:micromoles lipid), or more of lipid.

Liposomal preparations for use in the present disclosure include cationic (positively charged), anionic (negatively charged) and neutral preparations, with cationic liposomes particularly preferred. Cationic liposomes have been shown to mediate intracellular delivery of plasmid DNA, mRNA and purified transcription factors in functional form.

The liposomes can comprise multilammelar vesicles (MLVs), small unilamellar vesicles (SuVs), or large unilamellar vesicles (LUVs). The various liposome-nucleic acid complexes are prepared using methods known in the art.

The synthetic expression cassettes of interest may also be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG.

Furthermore, other particulate systems and polymers can be used for the in vivo or ex vivo delivery of the gene of interest. For example, polymers such as polylysine, polyarginine, polyornithine, spermine, spermidine, as well as conjugates of these molecules, are useful for transferring a nucleic acid of interest. Similarly, DEAE dextran-mediated transfection, calcium phosphate precipitation or precipitation using other insoluble inorganic salts, such as strontium phosphate, aluminum silicates including bentonite and kaolin, chromic oxide, magnesium silicate, talc, and the like, will find use with the present methods.

Recombinant vectors carrying a synthetic expression cassette of the present disclosure are formulated into compositions for delivery to the subject. These compositions may either be prophylactic (to prevent disease) or therapeutic (to treat disease). The compositions will comprise a “therapeutically effective amount” of the gene of interest such that an amount of the gene of interest can be produced in vivo in the individual to which it is administered. The exact amount necessary will vary depending on the subject being treated; the age and general condition of the subject to be treated; the severity of the condition being treated; the particular gene selected and its mode of administration, among other factors. An appropriate effective amount can be readily determined by one of skill in the art. Thus, a “therapeutically effective amount” will fall in a relatively broad range that can be determined through routine experimentation.

The compositions will generally include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Certain facilitators of immunogenicity or of nucleic acid uptake and/or expression can also be included in the compositions or coadministered, such as, but not limited to, bupivacaine, cardiotoxin and sucrose.

Accordingly, the present disclosure also provides for a method of treating and preventing at least one aging-associated condition comprising administering to a mammal in need of such treatment a therapeutically effective amount of a Cisd2 gene.

In some embodiments, the Cisd2 gene is selected from the group consisting of human Cisd2 gene and murine Cisd2 gene.

In some embodiments, the administering step comprises administering using a delivery vehicle. For example, the delivery vehicle may be a vector, a liposome, a polymer, a pharmaceutically acceptable composition, or a device which facilitates delivery of such delivery vehicle. In some embodiments, the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

In some embodiments, the therapeutically effective amount of a Cisd2 gene may be administered to a mammal in need of such treatment in a manner selected from the group consisting of intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intra-rectal administration, intra-tracheal injection; intra-nasal administration, intradermal administration, epidermal administration, oral administration and combinations thereof. In some embodiments, the mammal may be administered with multiple therapeutically effective amounts of the Cisd2 gene. In some embodiments, dosage treatment may be a single dose schedule or a multiple dose schedule. In other embodiments, the mammal may be administered with the Cisd2 gene in combination with another therapeutic. For example, the mammal may be a human.

The present disclosure further demonstrates a method of screening for compounds capable of modulating Cisd2 expression comprising providing a test compound; contacting a transgenic mouse of the present disclosure or a cell obtained or derived from the transgenic mouse the present disclosure with the test compound; and detecting whether the compound is capable of modulating Cisd2 expression.

The present disclosure further demonstrates a method of identifying an agent that modulates a characteristic of an age-associated condition comprising providing a transgenic mouse whose genome comprises a transgene encoding a mouse Cisd2, and which, compared with non-transgenic mice, exhibits a characteristic selected from the group consisting of decreased skin degeneration, decreased skeletal muscle degeneration, decreased neuronal degeneration, decreased mitochondrial damage, increased metabolism and increased lifespan; measuring said characteristic of the transgenic mouse; comparing the measured characteristic of (b) with that of a non-transgenic mouse; administering a test agent to the transgenic mouse of (a); and determining whether the test agent modulates the characteristic of an age-associated condition.

The present disclosure is further illustrated by the following specific examples. The examples are provided for illustration only and should not be construed as limiting the scope of the present disclosure in any way.

### EXAMPLES

### Example 1

### Generation of Cisd2 Transgenic Mice

The construction of the Pol II-Cisd2 transgenic plasmid is described herein. Specifically, the mouse Cisd2 (SEQ ID NO: 1) coding region was driven by the RNA polymerase II large subunit promoter (Pol II; NCBI Accession M14101 bases 1-712). The 0.33-kb synthetic intron and the 0.28-kb bovine growth hormone polyA signal (pA) were derived from the pIRES-EGFP plasmid (CLONTECH #6064-1). Two direct repeats of chicken HS4 insulator (NCBI Accession U78775 bases 10-1199) were placed downstream of the polyA signal to block positional effects. The Cisd2 transgenic mice were generated by pronucleus microinjection of C57BL/6 fertilized eggs. The genotypes of the mice were determined by PCR using tail DNA. The mice were bred in a specific pathogen-free facility; the animal protocol was approved by the Institutional Animal Care and Use Committee of the National Yang-Ming University.

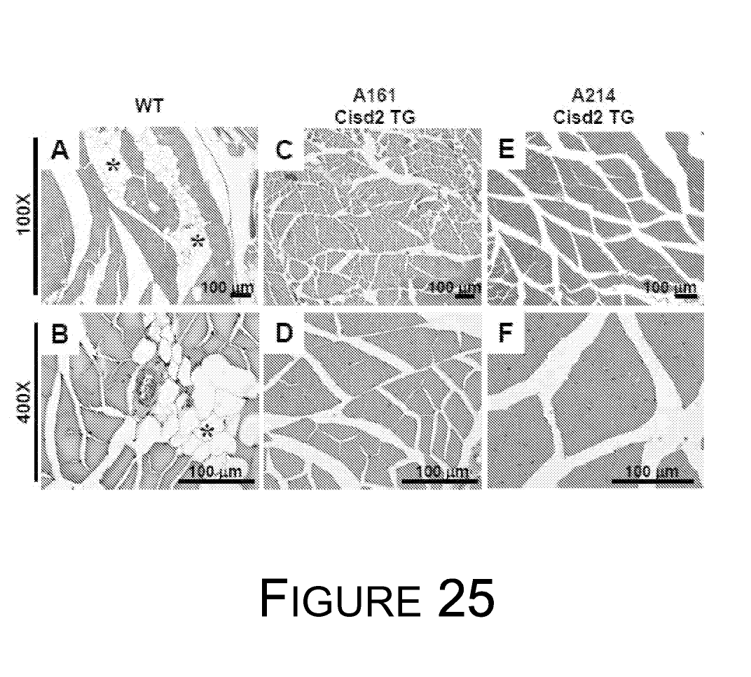

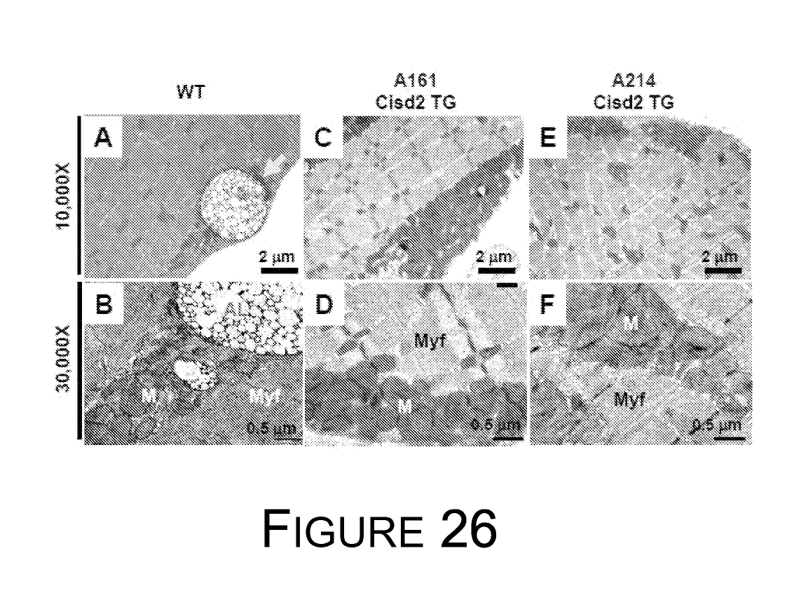

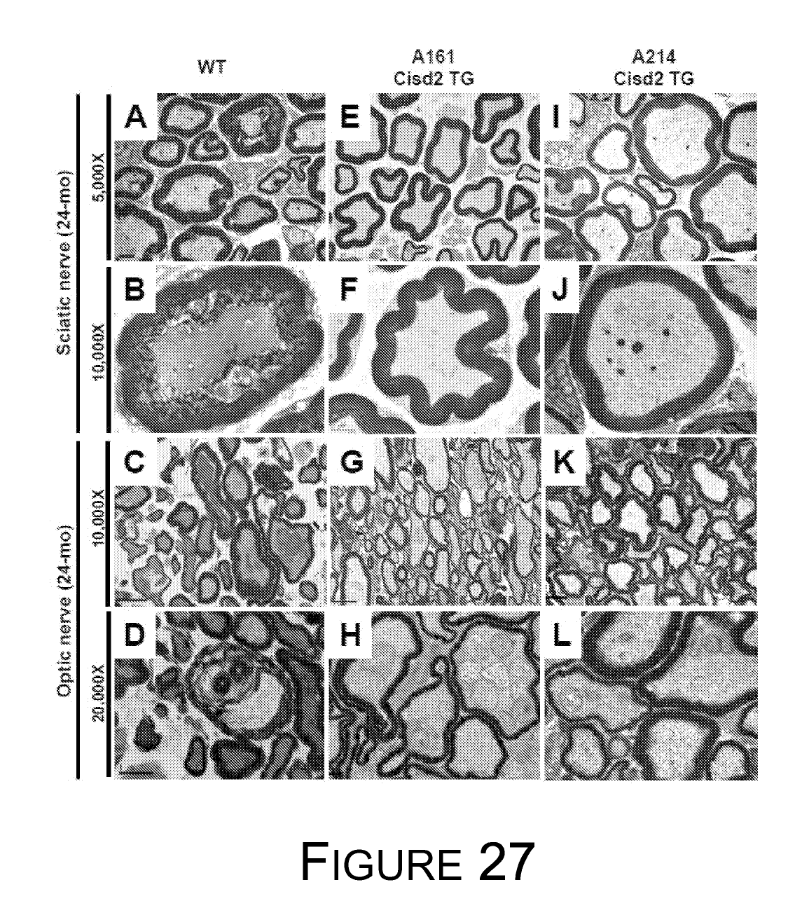

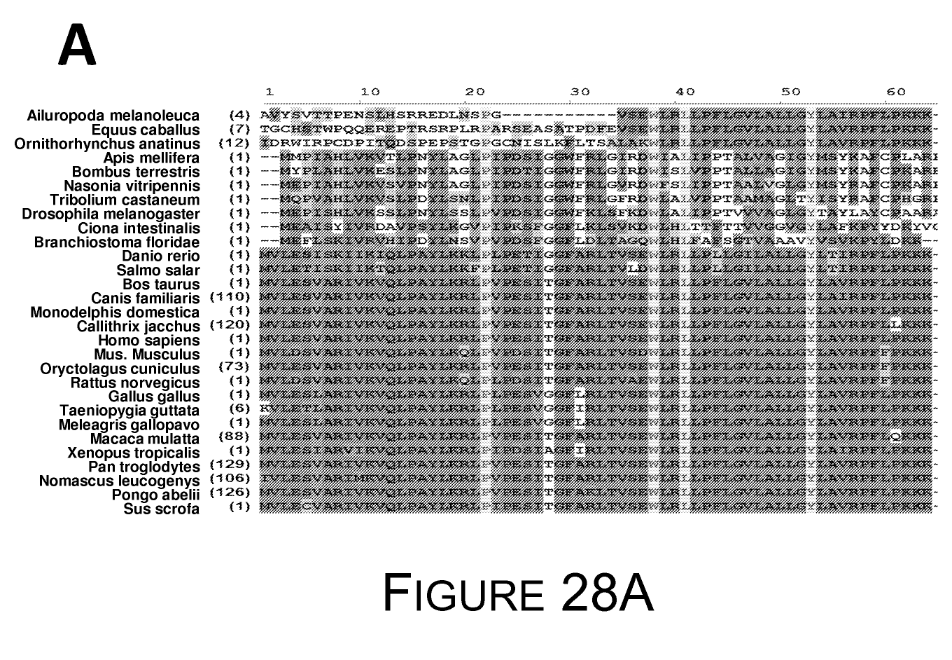

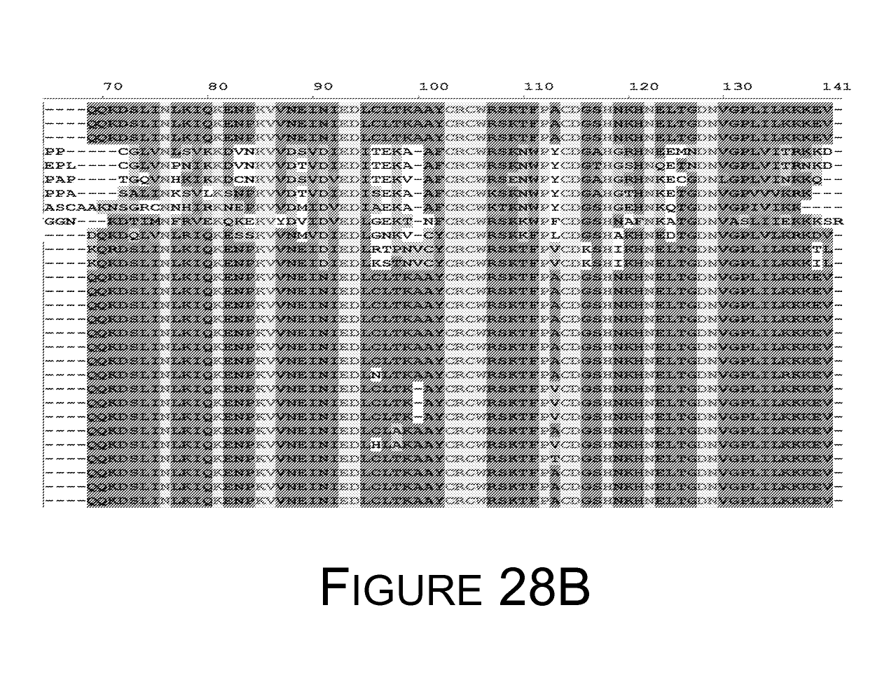

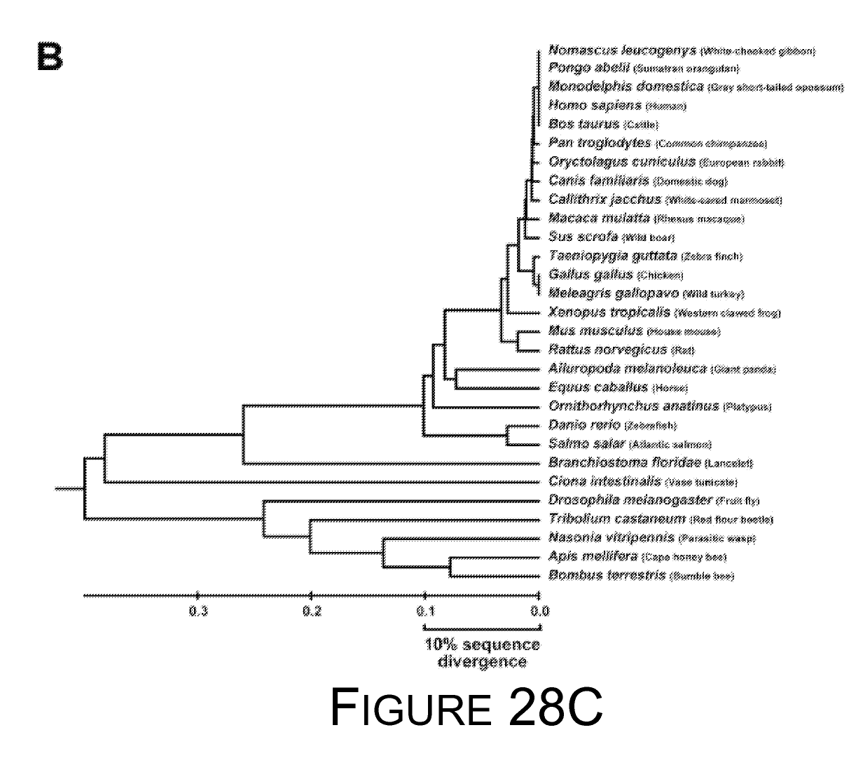

There are two independent transgenic mouse lines, A161 and A214; line A214 was created two years after the establishment of line A161. Both lines of the Cisd2 TG mice have a C57BL/6 background. Some of the results presented herein, including Figures and Table, were obtained using line A161. Results for the line A214 are presented in FIGS. 24-27 with the aim of confirming the reproducibility of the anti-aging phenotype mediated by Cisd2.

RNA Analysis

Total RNA was isolated from mouse tissues using TRIzol Reagent (Life Technology). Northern blot hybridization was performed as described previously (Sambrook and Russell, 2001). We execute real-time quantitative PCR with Roche LightCycler 480 Real-time PCR instrument, using TaqMan probe searched at Universal ProbeLibrary (Roche applied science) and LightCycler TaqMan Master (Roche applied science). All amplifications were carried out in triplicate for each RNA sample and primer set, and all real-time quantitative PCR measurements were done using RNA samples from three individual mice. The amount of total input cDNA was normalized using Hprt as an internal control.

Western Blotting

Tissue samples were homogenized in lysis buffer [20 mM Tris, pH7.4, 150 mM NaCl, 10 mM EDTA, 1% Triton X-100 with Complete protease inhibitor cocktail (Roche)] and denatured by boiling for 5 min. The extracted proteins were separated on a 13% SDS-polyacrylamide gel (BioRad) and electro-transfer to Amersham Hybond N+ membrane (GE Healthcare). The membranes were blocked with 5% (w/v) nonfat dry milk, incubated with primary antibody, washed and then detected using a Visualizer™ Kit (Upstate 64-201BP). The following antibodies were used for western blot analyses: TFAM (1:500; LifeSpan, LS-C30101); NDUFA9 (1:1000; Molecular Probes, A21344); SDHB (1:1000; Molecular Probes, A21345); UQCRC2 (1:1000; Molecular Probes, 459220); ATP5B (1:1000; Molecular Probes, A21351); Gapdh (1:5000, abcam ab9482). Preparation of rabbit anti-mouse Cisd2 polyclonal antibody was described previously (Chen et al., Genes Dev. 23, 1183-1194 (2009)).

Histopathology

Various mouse tissues were collected, fixed with 10% formalin buffered with phosphate and embedded in paraffin. Tissue sections (3-4 μm) were subjected to hematoxylin-eosin (H&E) and Masson's tirchome staining by standard procedures (Young and Heath, WHEATER'S Functional Histology, A Text and Colour Atlas, fourth ed. Churchill Livingstone, London (2003)).

Immunohistochemistry (IHC) Staining

IHC staining of perilipin was performed using paraffin-embedded skeletal muscle sections (3 μm). Muscle sections were soaked in antigen retrieval buffer containing 10 mM sodium citrate (pH 6.0) and heated in a microwave oven for 2×10 min. (Sunpentown SM-1220, 650W). The sections were then incubated with primary antibody against perilipin (1:100, Cell signaling D418 rabbit polyclonal antibody) at 40° C. for 18-24 hours, and detected by secondary antibody (Invitrogen Flour 570-conjugated goat anti-rabbit antibody). The sections were visualized by fluorescence microscope (OLYPUS BX51); the pictures were captured with DP Controller Ver. 3.1.1.267 software. Nuclei were counter stained with DAPI (4′-6-Diamidino-2-phenylindole; Sigma).

Transmission Electron Microscopy (TEM)

Various mouse tissues were fixed in a mixture of glutaraldehyde (1.5%) and paraformaldehyde (1.5%) in phosphate buffer at pH 7.3. They were post-fixed in 1% OsO₄, 1.5% potassium hexanoferrate, then rinsed in cacodylate and 0.2 M sodium maleate buffers (pH 6.0) and block-stained with 1% uranyl acetate. Following dehydration, the various tissues were embedded in Epon and sectioned for TEM as described previously (Kao et al. Atherosclerosis 116, 27-41 (1995)).

Water Repulsion

Mice were immersed in 37° C. water bath for 3 minutes and then placed on a paper towel to absorb excess water. Then the mice were exposed to ambient temperature; the body weight and temperature were recorded from 5 to 60 minutes after water immersion. The body temperature was detected by Microcomputer Thermometer MODEL 7000H (JENCO ELECTRONICS LTD).

Muscle Strength

The grip strength of a forelimb was determined using the Popular Model Digital Force Gauge (DS2, IMADA CO., LTD). Peak gripping force at the point of grip failure was recorded as grip strength. Each mouse underwent ten trials with 15 seconds of rest between each trial. There were more than three mice in each group, and three independent measurements were carried out for each mouse.

Oral Glucose Tolerance Test (GTT) and Insulin Tolerance Test (ITT)

Mice after a 10 hours fast (10 p.m.-8 a.m.) were orally administrated with glucose solution (1.5 g/kg body weight) using a feeding needle. Blood samples were collected from tail tips before (0 min) and after glucose load at the indicated time points. The blood glucose levels were measured using glucose test strips (LifeScan, Johnson & Johnson) and SureStep™ Brand Meter. Serum insulin levels were determined by an ELISA kit (Mercodia, Uppsala, Sweden). The insulin tolerance test was performed after a 2 hours fast (9 a.m.-11 a.m.) and involved an intraperitoneal injection of insulin (0.75 unit/kg body weight; Novolin human regular insulin, Novo Nordisk). There were three mice for each group and three independent measurements for each mouse.

Mitochondrial DNA (mtDNA) Copy Number

Relative mtDNA copy number was determined using quantitative PCR of total genomic DNA isolated from skeletal muscles. 50 ng DNA was subjected to real-time PCR using LightCycler-FastStar DNA Master SYBR Green I kit (Roche Applied Sciences). NADH dehydrogenase subunit 1 (ND1) gene (mitochondrial DNA encoded) and telomerase reverse transcriptase (Tert) gene (nuclear DNA encoded; this gene served as a calculated reference) were amplified with specific primers. The relative mtDNA copy number was measured using the RelQuant software (Roche Applied Sciences).

Mitochondrial DNA Deletion

Long PCR (13.6-kb) of mtDNA fragment for monitoring the integrity of mitochondrial genome was performed using genomic DNA isolated from liver.

Primers of long PCR: 5′-GCCAGCCTGACCCATAGCCATAATAT-3′ (SEQ ID NO: 2) and 5′-ATTAATAAGGCCAGGACCAAACCT-3′ (SEQ ID NO: 3). The relative percentage of the 13.6-kb long PCR and deleted mtDNA PCR signals was compared between wild-type and Cisd2 transgenic mice. PCR detection of D-17 deletion was performed using genomic DNA isolated from liver as described previously (Tanhauser et al., J. Biol. Chem. 270, 24769-24775 (1995)).

D-Loop Oxidative Damage

Oxidative damage of the D-loop of mtDNA was determined by monitoring the content of 8-OHdG. Genomic DNA isolated from skeletal muscle was treated with 8-hydroxyguanine DNA-glycosylase (OGG1) to remove the 8-OHdG residue and form an apuine site in the DNA template. The greater proportion of 8-OHdG in mtDNA, the less intact mtDNA will be left after OGG1 digestion; this will lead to a lower yield of the PCR products. Each of 500 ng DNA was first treated with or without 1 unit of hOGG1 at 37° C. for 1 hour, and these two groups of DNA were both amplified by quantitative real-time PCR using D-loop specific primers. The level of mtDNA D-loop damage is calculated as the efficiency of amplification of the OGG1-treated DNA group relative to that of the un-treated DNA group.

Measurement of Mitochondrial Oxygen Consumption

Mitochondria were isolated from mouse tissues as previously described (Chen et al., Genes Dev. 23, 1183-1194 (2009)). The oxygen consumption rate was measured using a 782 Oxygen Meter (Strathkelvin Instruments, Scotland, UK). An aliquot of 300 μl assay buffer (125 mM sucrose, 65 mM KCl, 2 mM MgCl2, 20 mM Na⁺, K⁺-phosphate buffer, pH 7.2) containing about 75 pg mitochondria were delivered into the closed chamber of the oxygen meter at 37° C. to measure the steady-state oxygen consumption rate of the mitochondria. To further estimate the respiratory function of mitochondria, we added 10 mM glutamate/malate (Sigma-Aldrich) as well as 1 mM ADP in order and measured the rate simultaneously in each stage. Finally, the oxygen consumption rate of isolated mitochondria was normalized by the weight of skeletal muscle.

Respiratory Enzyme Complex Activity

The activity of NADH dehydrogenase of Complex I was measured by following the reduction of potassium ferricyanide as described previously (Lu et al., J. Neurol. Sci. 177, 95-103 (2000)). An aliquot of total protein lysate extracted from skeletal muscle was incubated with the assay mixture (2 mM KCN, 0.5 mM β-NADH, 20 mM K₂HPO₄ at pH 7.4). After addition of K₃Fe(CN)₆ to the mixture, the change in the absorbance at 420 nm was recorded on a UV/visible spectrophotometer. On the other hand, the rotenone, a specific Complex I inhibitor, was added in the reaction to measure the NADH dehydrogenase activity excluding Complex I. By subtracting this from total activity, the Complex I activity was obtained. The activity assays of Complex I-III, Complex IIIII, and Complex IV were performed as described previously (Wei et al., Ann. N.Y. Acad. Sci. 854, 155-170 (1998)). The activities of NADH cytochrome c reductase (NCCR; which represents Complex I-III activity) and succinate cytochrome c reductase (SCCR; which represents Complex II-III activity) were measured by following the reduction of exogenous oxidized cytochrome c. An aliquot of 20˜50 μg submitochondrial particles (SMP) was pre-incubated with the assay buffer (1.5 mM KCN, 50 mM K₂HPO₄, pH 7.4) containing β-NADH or succinate at 37° C. for 15 min. After addition of cytochrome c to the mixture, the change in the absorbance at 550 nm was recorded on a UV/visible spectrophotometer. Cytochrome c oxidase (CCO; which represents Complex IV) activity was determined by following the oxidation of exogenous reduced cytochrome c. An aliquot of 20˜50 μg SMP was pre-incubated in the assay buffer (5 mM K₂HPO₄, pH 7.4) at 30° C. for 10 min. After addition of ferrocytochrome c to the assay mixture, the change in absorbance at 550 nm was recorded on a UV/visible spectrophotometer.

Whole-Body Energy Metabolism

Whole-body energy metabolism was measured by indirect calorimetry. TSE calorimetry Module of the LabMaster System was used to determine oxygen consumption rate (VO₂), carbon dioxide production rate (VCO₂), and energy expenditure (heat) in small laboratory animals. Mice were monitored over 24 h in indirect calorimetry.

Statistics

Results are presented as means±SD. Comparisons between two groups were done using a Student's t test. Mouse survival rates were calculated by the Kaplan-Meier method, and differences in the survival of different groups of mice were determined by the log-rank (Mental-Cox) test. When analyzing statistical differences between different groups of mice, p<0.05 was considered significant.

### Example 2

### Cisd2 Promotes Longevity

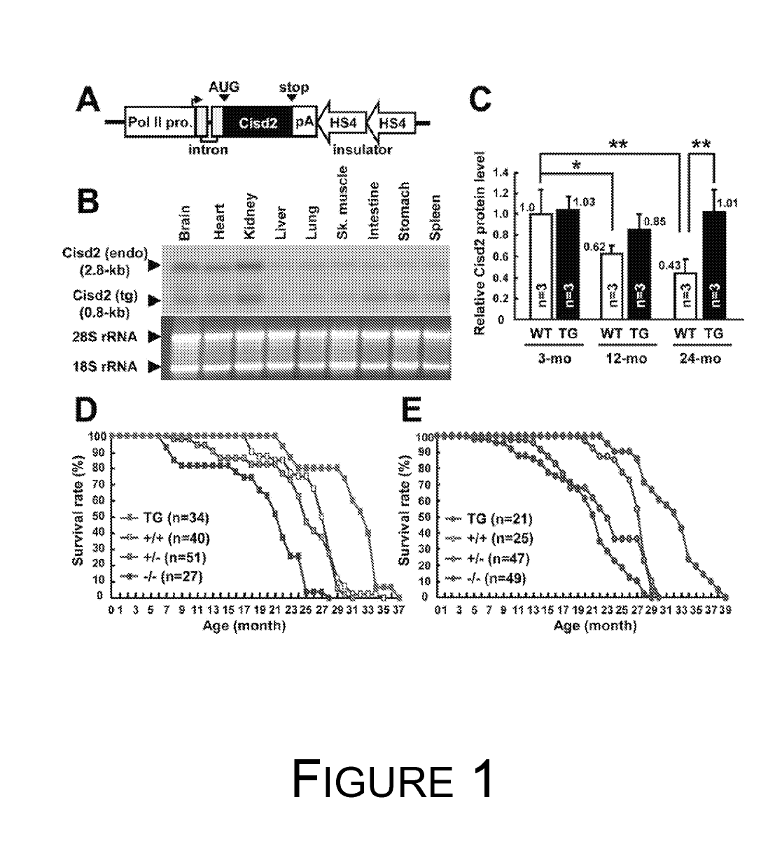

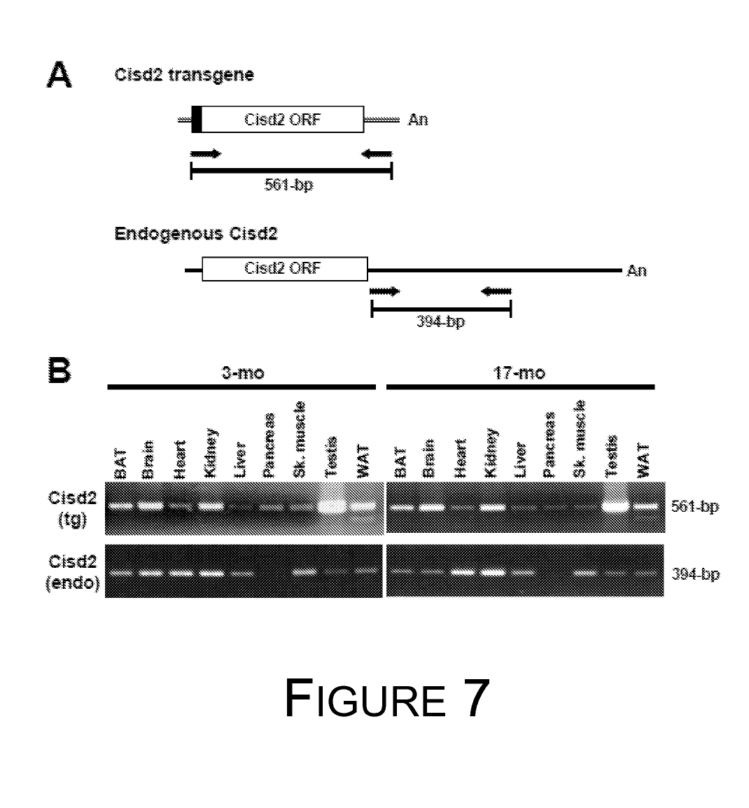

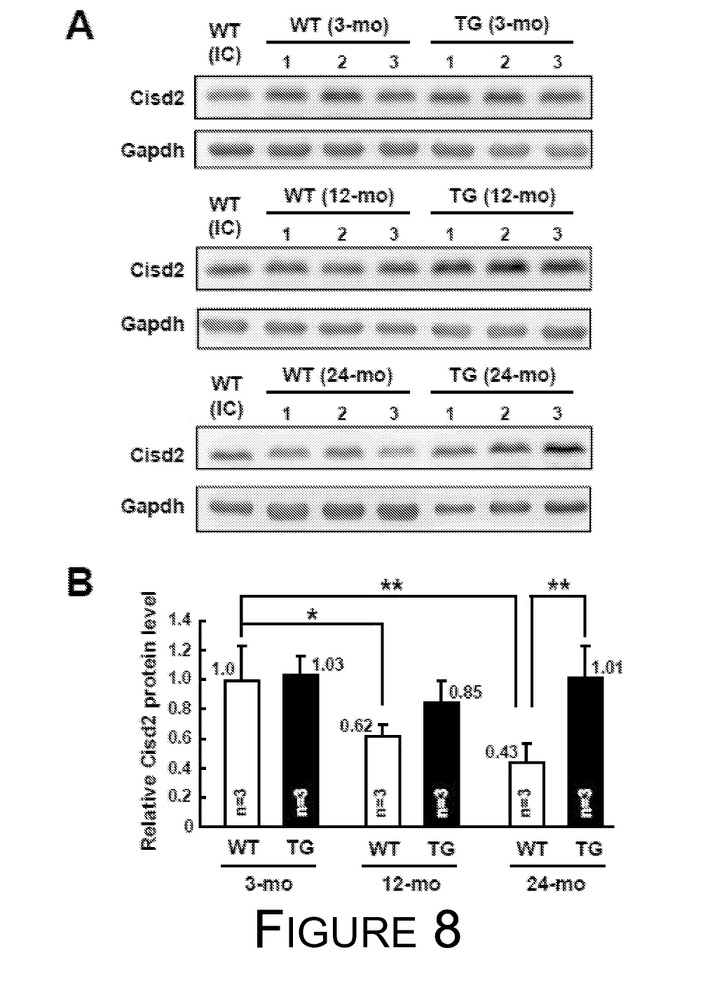

To study if enhanced expression of Cisd2 can extend lifespan, delay aging and help to retain the functional abilities lost with age, we generated Cisd2 TG mice carrying the Cisd2 coding region controlled by the RNA polymerase II large subunit (Pol II) promoter in a C57BL/6 mouse background (FIG. 1A). RNA analyses revealed a similar pattern for the endogenous and transgenic Cisd2 mRNA expression using Northern blot and RT-PCR (FIG. 1B; FIG. 7). In wild-type (WT) mice, the levels of Cisd2 decrease in an age-dependent manner during aging. In the present study we show that there is an average 38% and 57% decrease in the Cisd2 protein level in skeletal muscle of WT mice at middle age (12-mo) and old age (24-mo), respectively, compared to young (3-mo) mice; however, in Cisd2 TG mice, there is a persistent expression level of the Cisd2 protein from young (3-mo) through middle age (12-mo) to old age (24-mo) (FIG. 1C; FIG. 8). It appears that the transgenic Pol II promoter exhibited constitutive activity during aging when driving Cisd2 expression.

An extended lifespan was evident for both sexes of the Cisd2 TG mice without any statistically significant sex differences (Table 1). In males (FIG. 1D; Table 1), the median lifespan in the Cisd2 TG mice was increased by 5.25 months (19.4%, from 27 months to 32.25 months) relative to WT mice (p<0.001), while the maximum lifespan (mean lifespan of the oldest 10% within a cohort) was increased by 3.66 months (11.7%, p=0.043). In females (FIG. 1E; Table 1), the median lifespan in Cisd2 TG mice was increased by 5.1 months (18.7%, from 27.15 to 32.25 months) relative to WT mice (p<0.001), while the maximum lifespan was increased by 8.75 months (29.6%, p=0.032).

TABLE 1
Extended Median and Maximum Lifespan of the Cisd2 TG Mice
Table 1. Extended Median and Maximum Lifespan of the Cisd2 TG Mice

| | | | | | | |
|---|---|---|---|---|---|---|
| Genotype | Median | Mean | Maximum | Minimum | Oldest 10% | Youngest 10% |

Male

| | | | | | | | |
|---|---|---|---|---|---|---|---|
| Cisd2 TG | 32.25 | 30.72 ± 1.18 | 36.73 | 21.73 | 34.86 ± 1.62 | 22.51 ± 0.77 | 34 |
| WT | 27 | 25.74 ± 0.62 | 34 | 17.06 | 31.20 ± 1.93 | 17.31 ± 0.21 | 40 |

Female

| | | | | | | | |
|---|---|---|---|---|---|---|---|
| Cisd2 TG | 32.25 | 31.22 ± 0.96 | 39 | 22.2 | 38.35 ± 0.92 | 22.94 ± 1.04 | 21 |
| WT | 27.15 | 26.28 ± 0.41 | 29.8 | 19.13 | 29.60 ± 0.28 | 19.57 ± 0.62 | 25 |

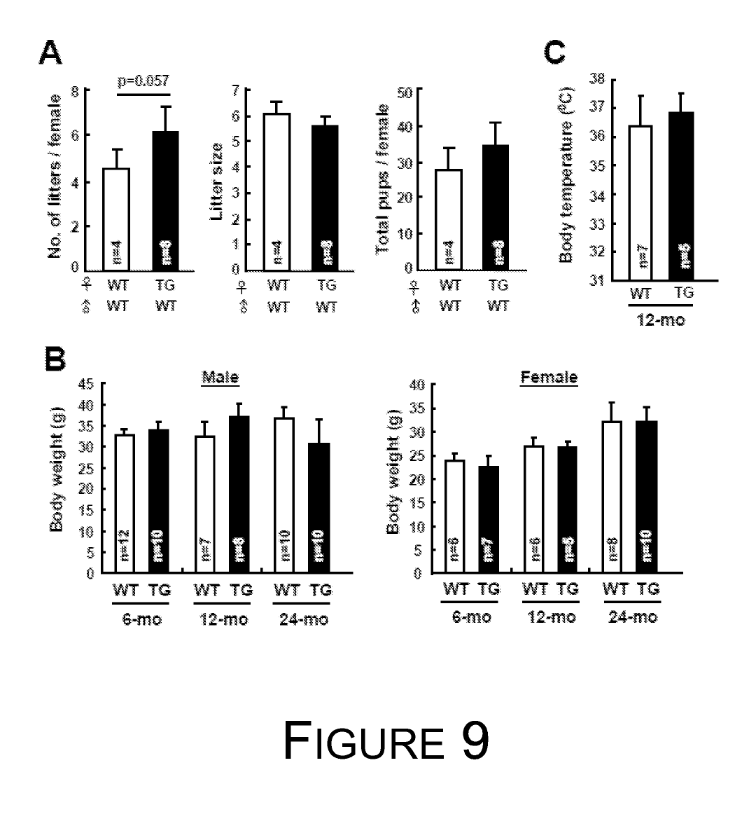

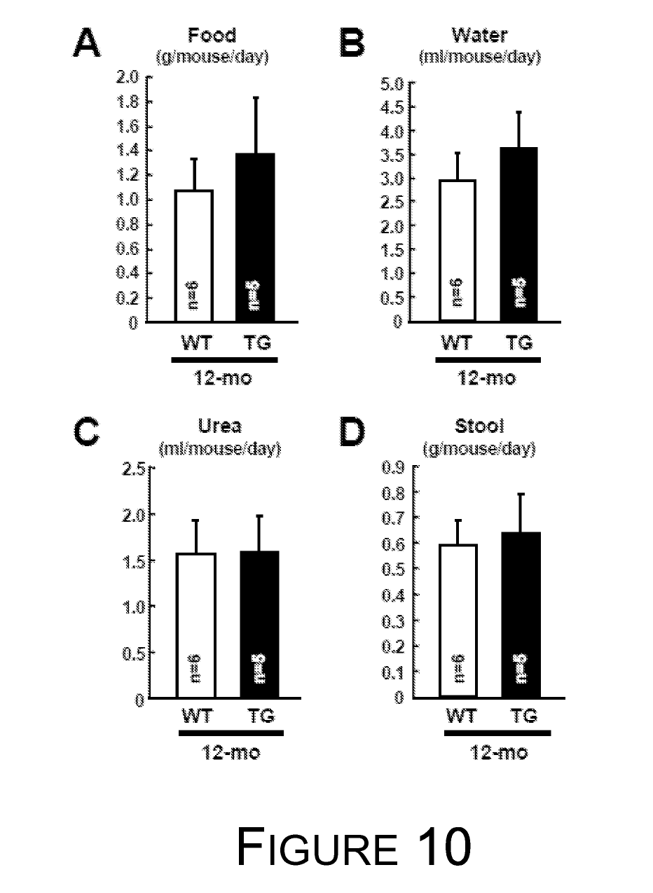

According to evolutionary theory, in particular the disposable soma theory, the maximum fitness of an organism is a trade-off between fertility and longevity. The long-lived Klotho TG mice provide an example of this inverse correlation between fertility and lifespan. Furthermore, the cost of reproduction is higher for females than for males due to the energetic and nutritional requirements of pregnancy and lactation. To detect possible effects of constitutive Cisd2 expression on reproductive phenotypes, we performed a breeding test using Cisd2 TG females. The numbers of litters and pups as well as the litter size were recorded from 2-month old (2-mo) to 12-mo for each female. Our results revealed no obvious difference in female fertility between the WT and Cisd2 TG mice (FIG. 9A). In addition, there was no obvious phenotypic effect on body weight or body temperature when the Cisd2 TG mice were compared to WT mice (FIGS. 9B, 9C). Additionally, we measured the metabolic indices of the Cisd2 TG mice, including intake of food and water and generation of urine and stool. No significant difference in these metabolic indices was observed (FIG. 10). These results indicate that Cisd2 modulates lifespan through mechanisms that are likely to be independent of growth, food intake and reproduction.

### Example 3

### Cisd2 Delays Skin Aging

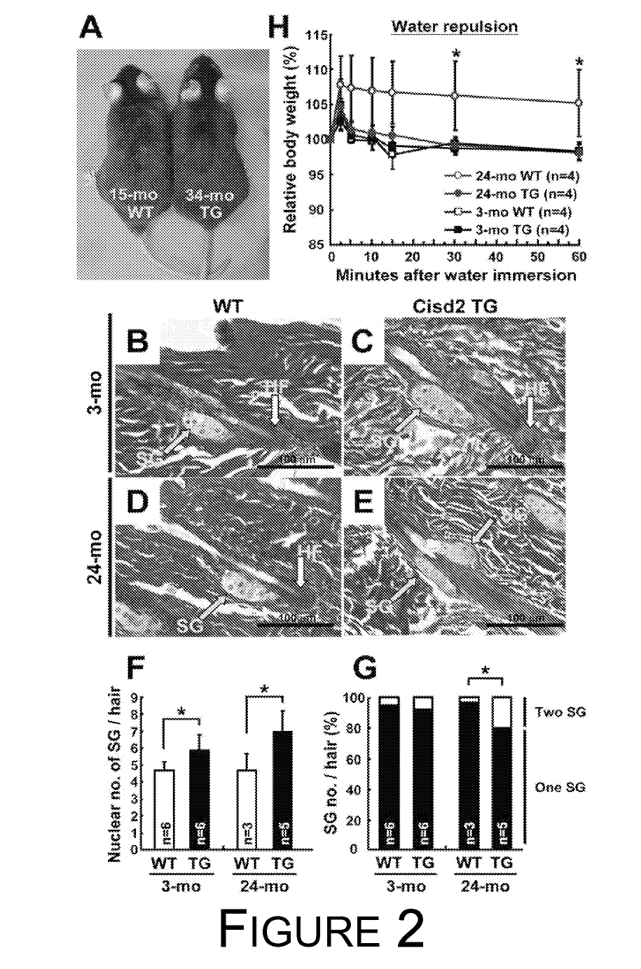

Age-associated structural and functional changes are more visibly evident in the skin than in any other organ in mammals. Interestingly, the fur of a very old (34-mo) Cisd2 TG mouse appeared to have less grey hairs (de-pigmentation) and to display a more prominent sheen than that of a middle-age (15-mo) WT mouse (FIG. 2A). Histological examination (FIGS. 2B-2E) and quantification (FIG. 2F) revealed that age-dependent atrophy of the sebaceous glands, which are the lipid-producing structures associated with the hair follicles, was significantly delayed in Cisd2 TG mice at 24-mo. In addition, the proportion of individual hair follicles associated with two sebaceous glands was significantly increased in the Cisd2 TG mice at 24-mo (FIG. 2G).

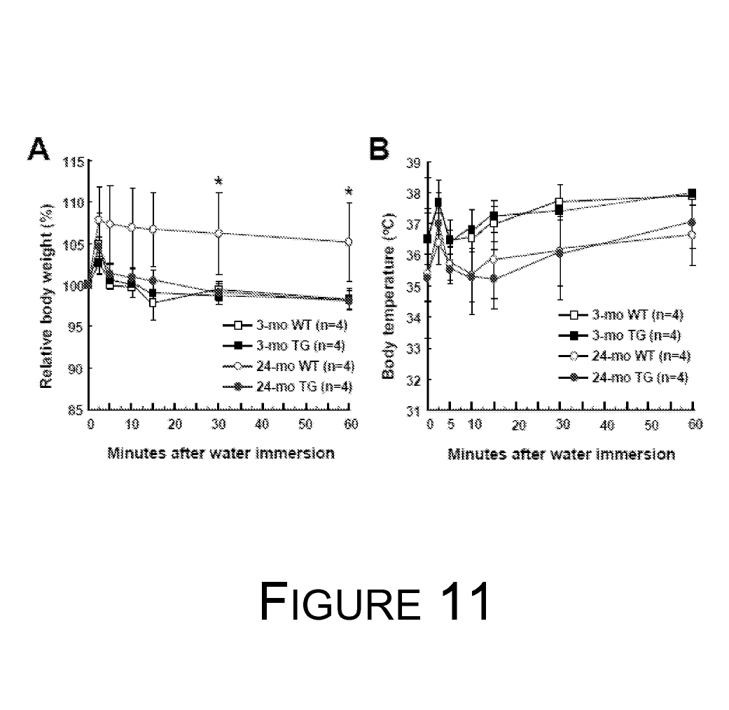

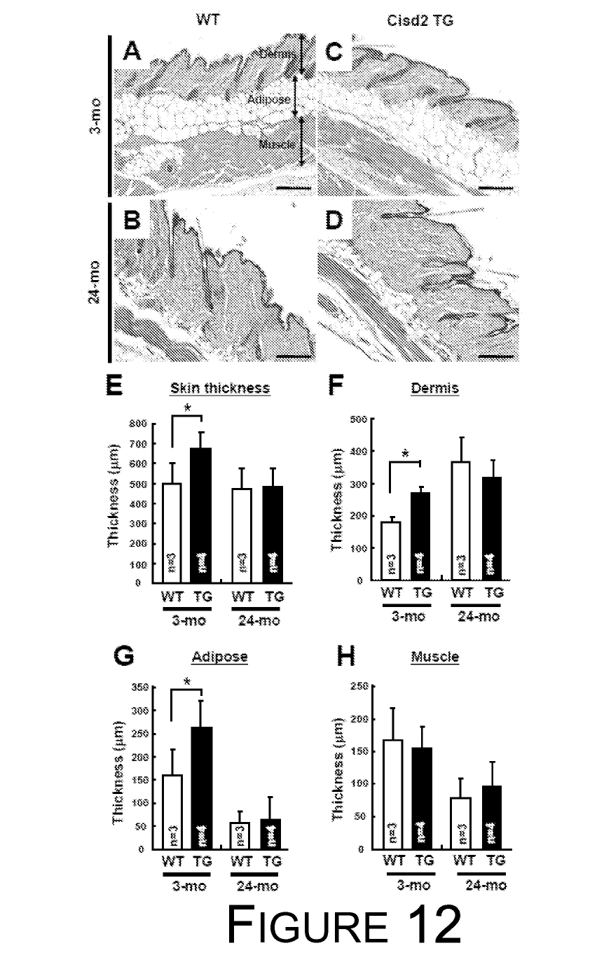

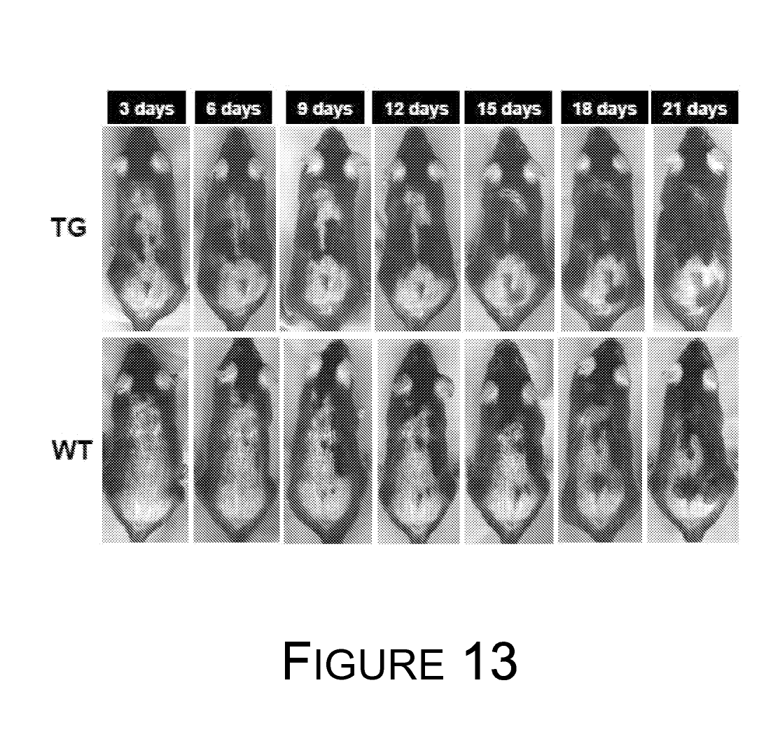

Sebaceous glands secrete lipids (sebum) that coats the hair; in furry mammals, these lipids play important roles in water repulsion and thermoregulation. Sebaceous glands change as skin ages and therefore we tested the ability of different ages of mice to repel water and maintain body temperature when wet. Although the core body temperature after water immersion showed no significant difference between age-matched WT and Cisd2 TG mice (FIG. 11), the water repulsion test revealed an interesting difference. Five minutes after water immersion, young mice at 3-mo from both the WT and Cisd2 TG groups were nearly dry (FIG. 2H). As expected, old mice from the 24-mo WT group, which displayed sebaceous gland atrophy, exhibited impaired water repulsion (FIG. 2H). Remarkably, the ability of old (24-mo) Cisd2 TG mice to repel water was similar to that of young (3-mo) mice (FIG. 2H). Skin aging is also associated with a decrease in skin thickness, which is mainly due to atrophy of the subcutaneous fat and muscle; however, this phenotype was observed in both WT and Cisd2 TG mice at 24-mo (FIG. 12). Nevertheless, an increase in hair re-growth rate was observed for the Cisd2 TG mice when middle aged (12-mo) (FIG. 13). When taken together, these results revealed that skin aging seems to be significantly delayed by the persistent expression of Cisd2 and, most obviously, Cisd2 alleviates sebaceous gland atrophy during skin aging.

### Example 4

### Cisd2 Delays Muscle Aging and Improves Insulin Sensitivity

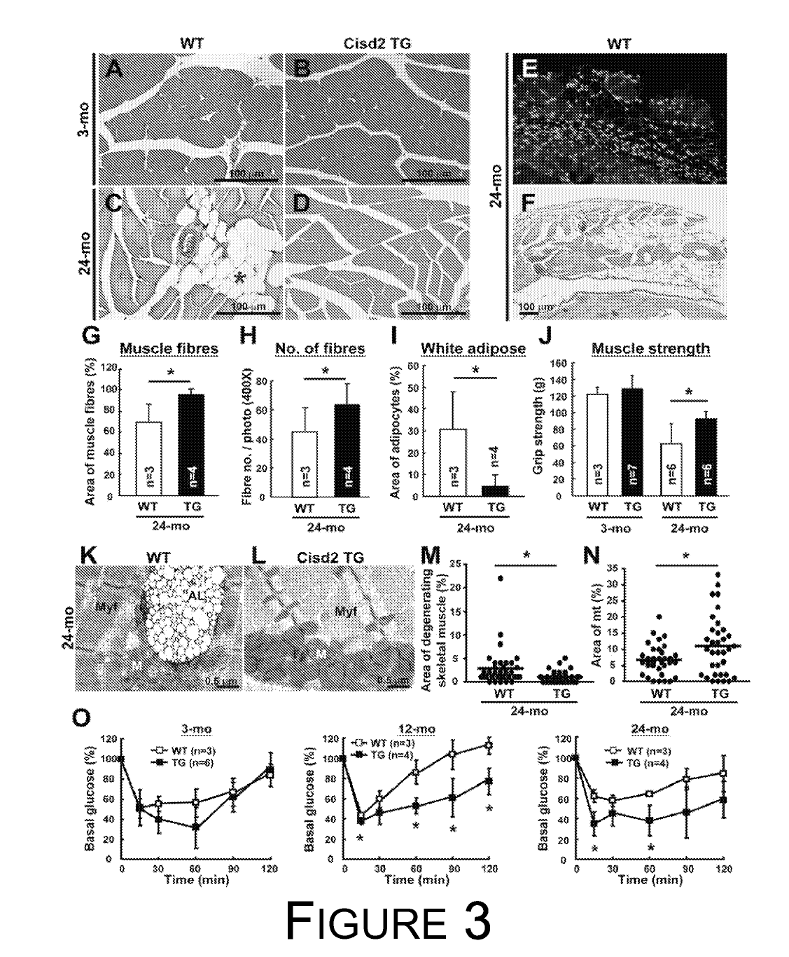

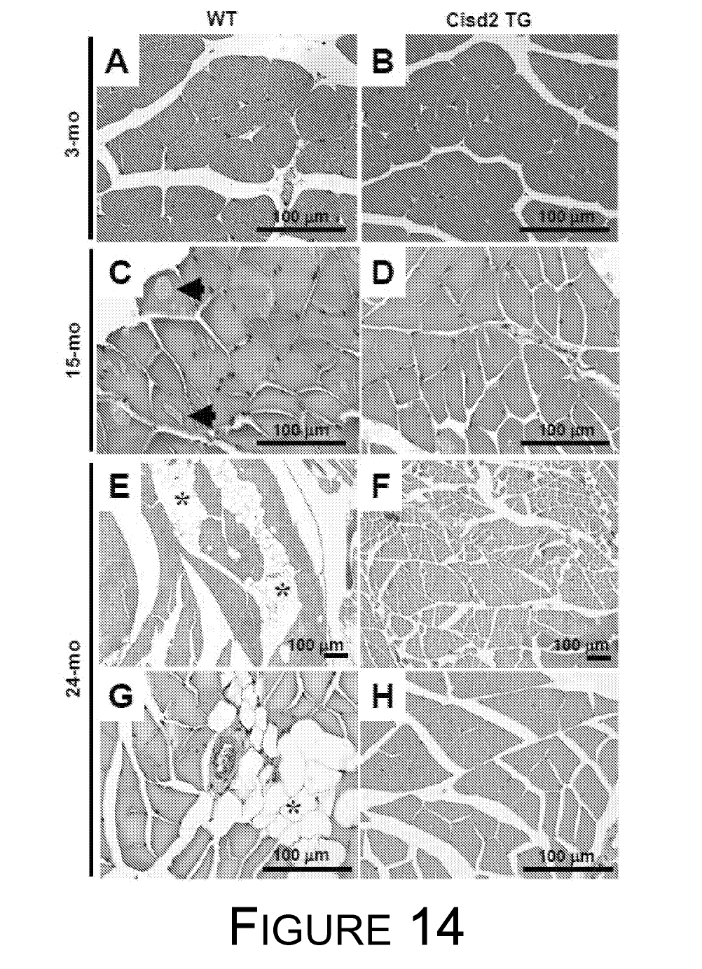

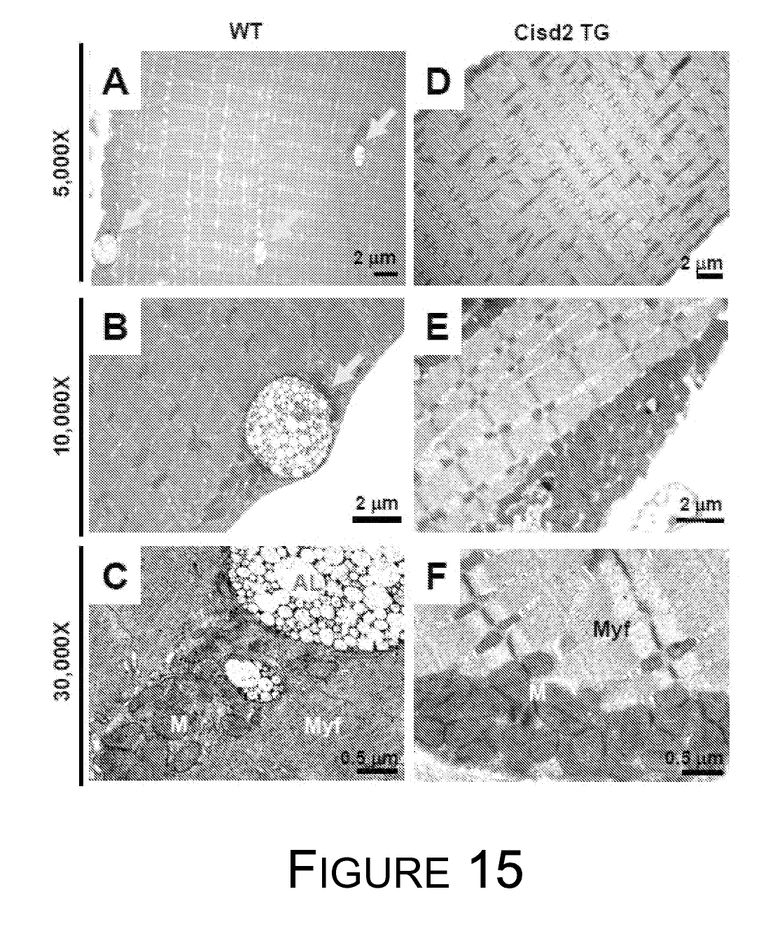

Muscle strength declines with aging. Quantitative loss of muscle mass, namely sarcopenia, is the most important factor underlying this phenotype. Sarcopenia is accompanied by increased muscle fat infiltration and the accumulation of fat mass in aged muscle has been shown to be a predictor of subsequent functional loss and disability in humans. Strikingly, constitutive Cisd2 expression has a profound effect and protects skeletal muscles from age-dependent mass losses and prevents fat infiltration (FIGS. 3A-3F; FIG. 14). Quantification of the phenomena revealed that there was a significant increase in fiber size (FIG. 3G) and fiber number (FIG. 3H), and a significant decrease in lipid infiltration (FIG. 31) in the Cisd2 TG mice at 24-mo. Furthermore, functional muscle strength improvement in the Cisd2 TG mice was demonstrated using a grip strength meter (FIG. 3J). A detailed transmission electron microscopy (TEM) examination further revealed that Cisd2 indeed protects from age-associated degeneration the mitochondria within and the ultrastructure of skeletal muscle, in Cisd2 TG mice, including myofilaments; this is in contrast to the overt mitochondrial degeneration, which is accompanied with autophagy, in the aged muscles of WT mice at 24-mo (FIGS. 3K-3N; FIG. 15).

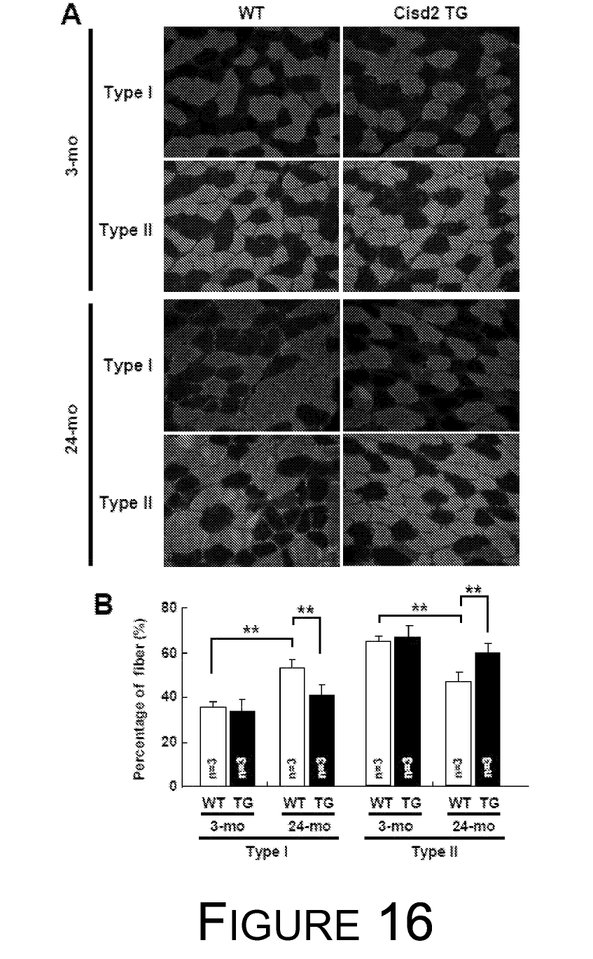

There are two types of fibers in mammalian skeletal muscles. These are type I (slow-twitch) fibers, which are enriched in mitochondrial content and are dependent on oxidative phosphorylation and type II (fast-twitch) fibers, which are primarily glycolytic and are susceptible to fatigue. Previous studies of the elderly human skeletal muscle have revealed that there is a selective amplification of type I fibers with age, while there is a parallel atrophy of the type II fibers. Additionally, an apparent grouping of both types of fibers had been observed in the elderly humans; both fiber types seem to be distributed in clusters rather than in a random fashion, which is the commonly observed pattern in young muscle. This age-associated fiber age did not change the fiber distribution and ratios of type I to type II fibers in Cisd2 TG mice (FIG. 16). These results provide independent evidence that Cisd2 helps to maintain normal function and physiology in skeletal muscles during aging.

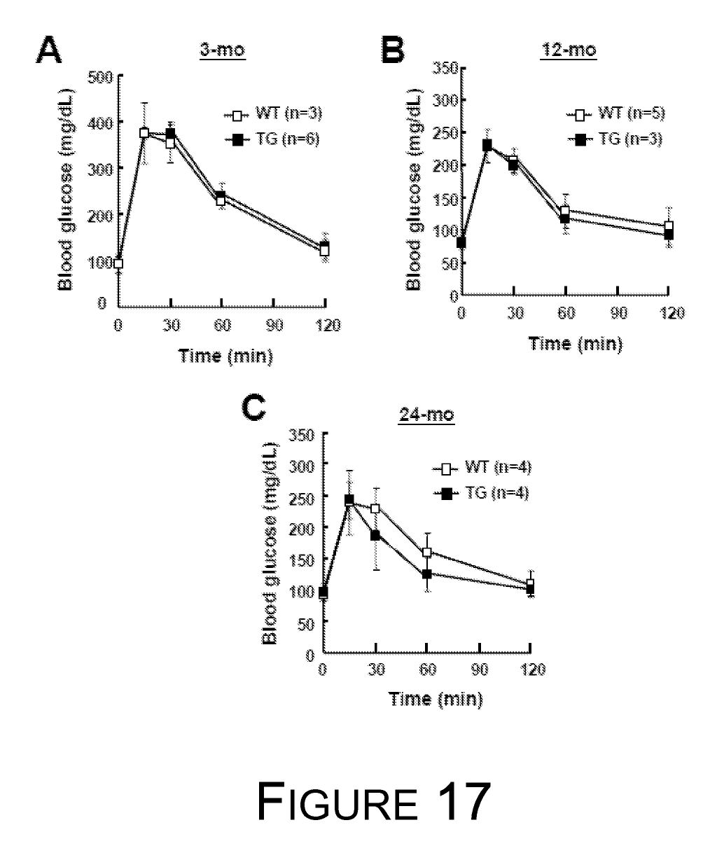

There is evidence that insulin sensitivity declines during aging in humans and that the elderly showed marked insulin-resistance, that is impaired insulin sensitivity, compared to young controls. Previous studies have shown that insulin resistance in elderly is paralleled by increased fat accumulation in muscles along with structural and functional abnormalities of muscular mitochondria. These results thus support the hypothesis that an age-associated decline in mitochondrial function contributes to insulin resistance in humans. In our mouse study, we sought to determine whether protection from the age-associated damages to muscle mitochondria would result in protection from age-associated insulin resistance. To this end we performed oral glucose tolerance tests (GTT) and insulin tolerance tests (ITT) on the WT and Cisd2 TG mice. Our results revealed no obvious difference in GTT (FIG. 17). However, there was a significant difference in ITT between the WT and Cisd2 TG mice with the Cisd2 TG mice showing increased insulin sensitivity compared with the WT mice at middle (12-mo) and old age (24-mo) (FIG. 30). This indicates that the Cisd2 protein indeed is able to protect mice from the progressive decline in insulin sensitivity during aging.

### Example 5

### Cisd2 Delays Neuron Aging

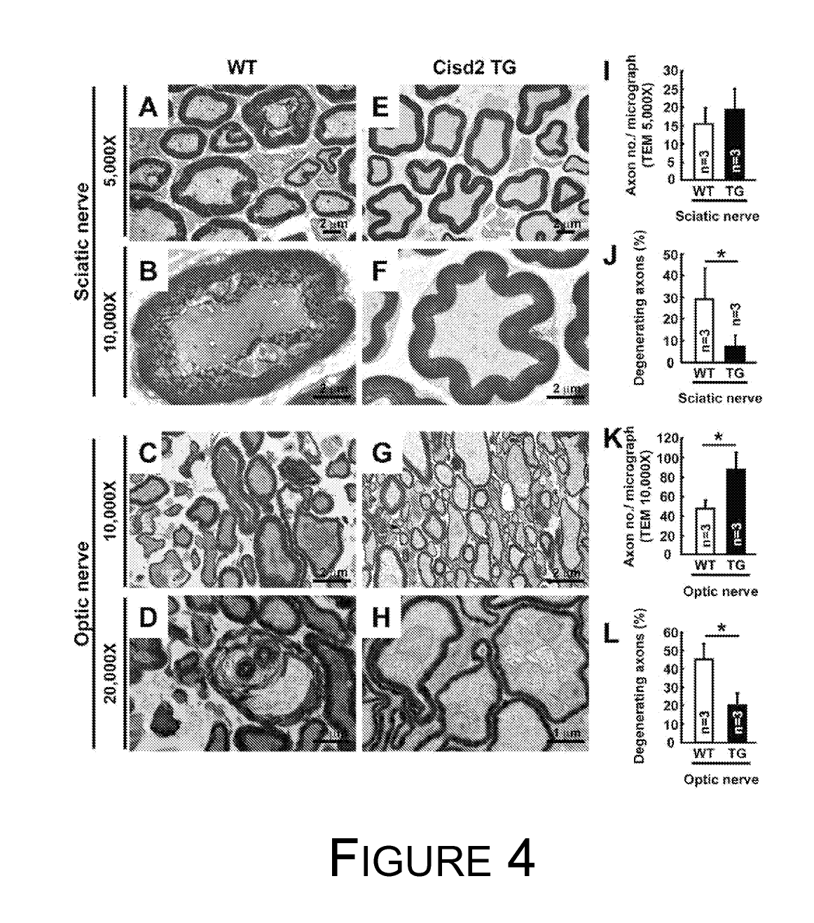

Morphologically, the non-myelinated axons and myelinated axons, which are enveloped by a myelin sheath formed by the fusion of many layers of plasma membrane from Schwann cells, can be identified using TEM micrography (FIG. 4). Notably, considerable age-associated degeneration of the non-myelinated and myelinated axons, and the disintegration of the myelin sheath were detected in the sciatic nerve (FIGS. 4A, 4B) and optic nerve (FIGS. 4C, 4D) of aged WT mice at 24-mo. Interestingly, the persistent expression of Cisd2 appears to protect the TG mice from the age-associated damage to both the sciatic nerve (FIGS. 4E, 4F) and optic nerve (FIGS. 4G, 4H). Furthermore, neuron degeneration in the aged WT mice seems, in turn, to lead to a decrease in neuron density and this is supported by our quantification. These results mean that there are striking differences in the numbers of myelinated axons in the sciatic and optic nerves when WT and Cisd2 TG mice are compared (FIGS. 4I-4L).

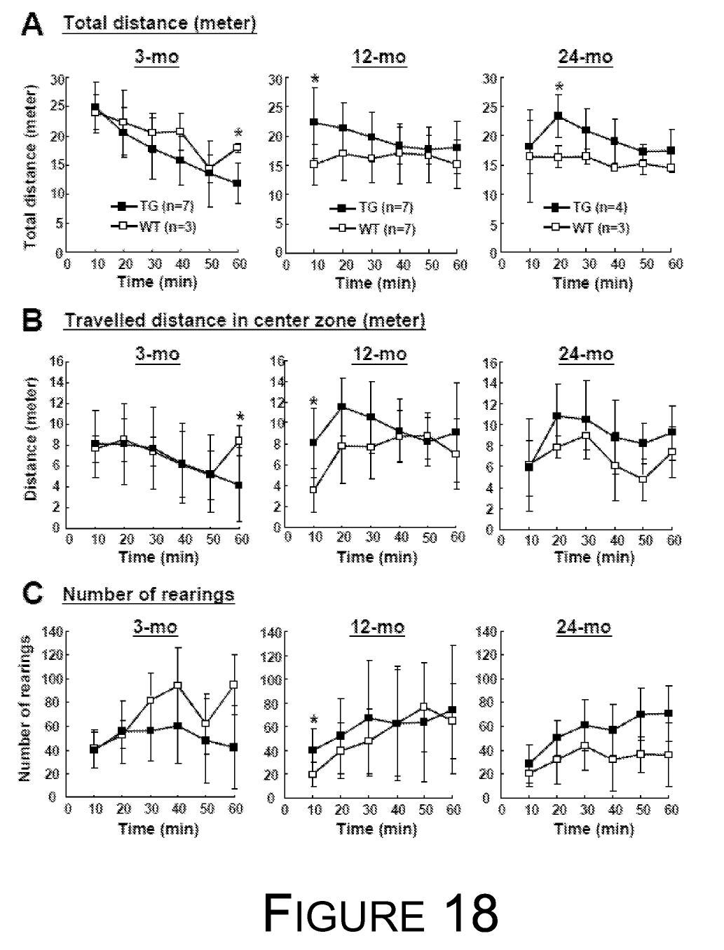

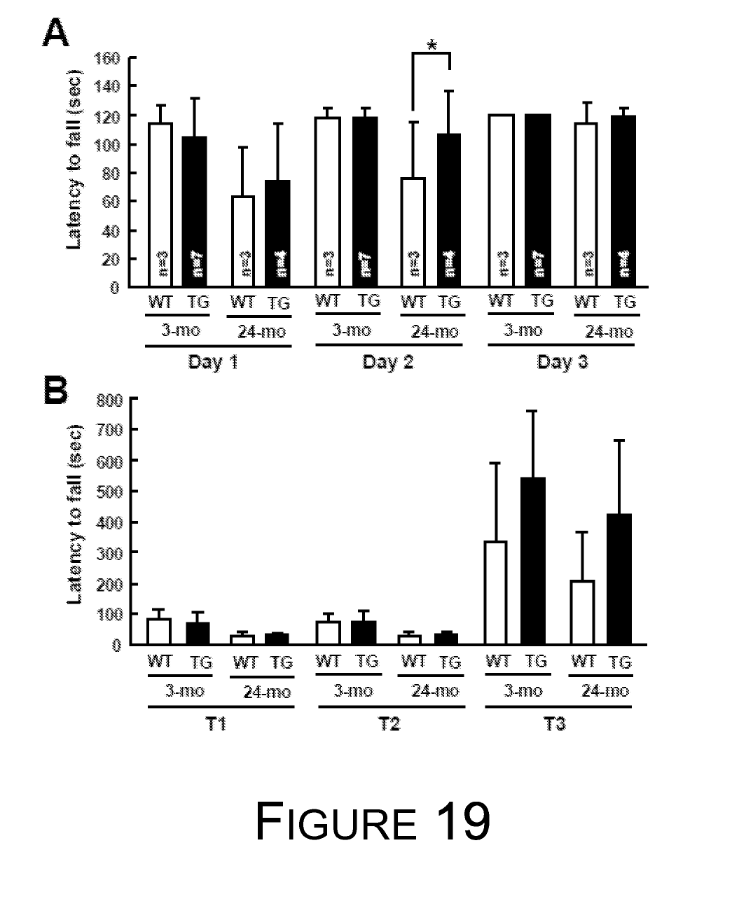

To examine motor functions in older mice, we assessed behavior using open field locomotion and rotarod test. There was a trend toward better motor function in the Cisd2 TG mice compared to the age- and sex-matched WT mice at 24-mo, but this effect was not statistically significant (FIGS. 18, 19). Thus it seems that the age-associated pathological changes brought about by constitutive Cisd2 expression are the most consistently difference in neurons between WT and Cisd2 TG mice, particularly at the ultrastructural level.

### Example 6

### Cisd2 Protects Mitochondria from Age-Associated Damage, Reduces The Age-Associated Declines in Mitochondrial Function and Whole-Body Energy Metabolism

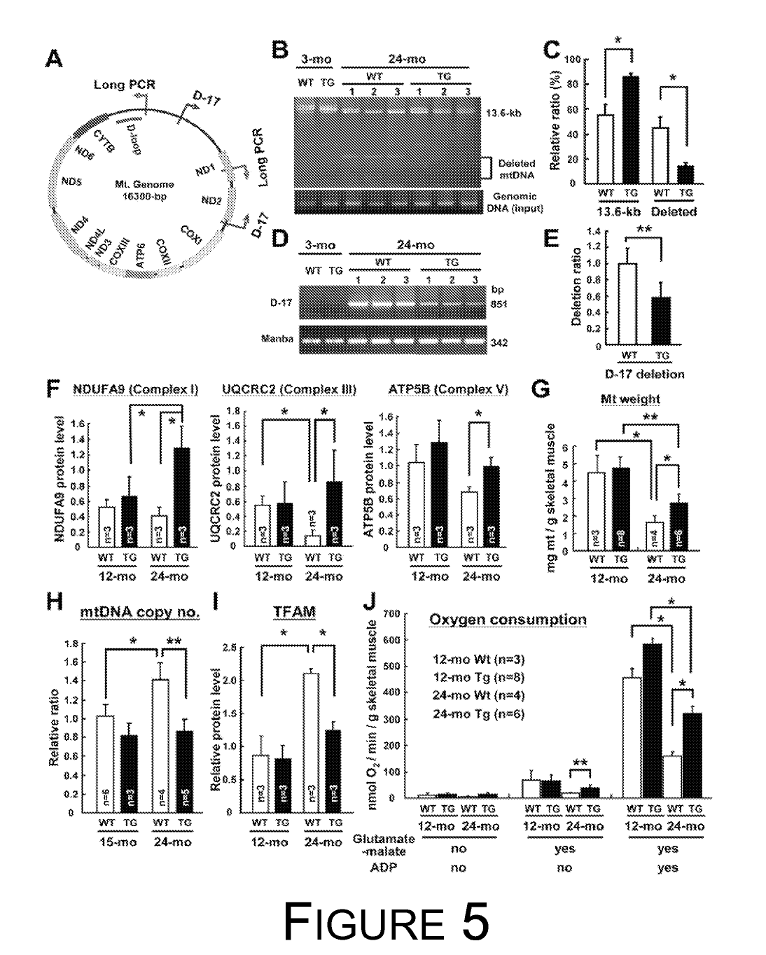

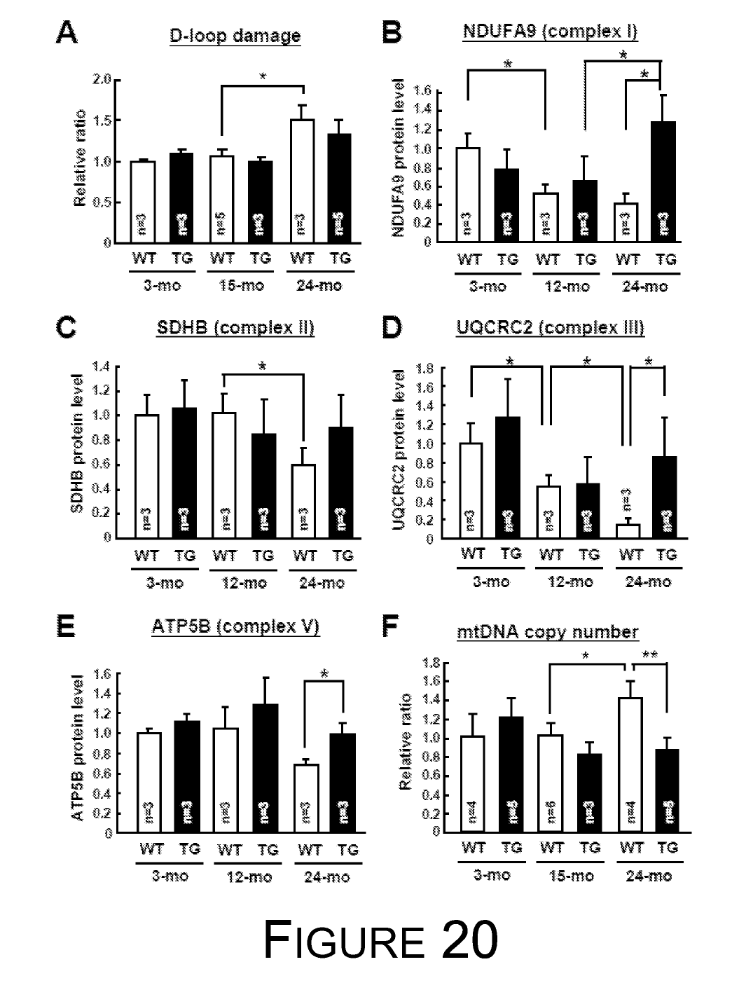

There is progressive damage to mitochondrial DNA (mtDNA) during aging. To study whether Cisd2 is able to protect mitochondria from age-associated genomic damage, we examined mtDNA integrity by long PCR (13.6-kb) of mtDNA; this amplification covers >83% mitochondrial genome (FIG. 5A). DNA polymerase amplifies only undamaged templates, therefore any damage to the mtDNA (such as strand breaks, abasic sites, and certain types of oxidative lesions) will block the progression of the DNA polymerase, thus decreasing amplification; in addition, internal mtDNA deletions may yield shorter PCR fragments that can be visualized by gel electrophoresis. Indeed, we detected a reduction in the amplification of the 13.6-kb fragment and an increase in the number of shorter PCR fragments in the old (24-mo) WT mice compared with the young (3-mo) mice (FIGS. 5B, 5C). In contrast, the old (24-mo) Cisd2 TG mice were protected from this age-associated increase in mtDNA damage (FIGS. 5B, 5C). Additionally, PCR amplification of the D-17 deletion, which covers the ND1 and ND2 genes (Tanhauser and Laipis, 1995), revealed that Cisd2 attenuates this age-associated mtDNA deletion in the old (24-mo) Cisd2 TG mice (FIGS. 5D, 5E). Nonetheless, there was no significant difference in D-loop oxidative damage (FIG. 20A).

Furthermore, examination of nuclear-encoded mitochondrial proteins involved in the oxidative phosphorylation revealed a significant increase in the levels of NDUFA9 (a component of the complex I), UQCRC2 (a component of the complex III) and ATP5B (a component of the complex V) in the skeletal muscle of old (24-mo) Cisd2 TG mice compared to WT controls (FIG. 5F). These proteins show an age-dependent reduction during aging and this reduction was obviously attenuated in the Cisd2 TG mice (FIGS. 20B-20E).

Mitochondrial transcription factor A (TFAM) is an mtDNA binding protein essential for the maintenance, replication and transcription of mtDNA. Previous studies have revealed a close association between changes in the level of TFAM and mtDNA copy number during aging. TFAM and mtDNA content are increased in the skeletal muscle of aged humans and aged rats; this phenomenon is likely to be a compensatory response that acts through nuclear-mitochondrial cross-talk. In our mouse study, we found the total amount of mitochondria per gram of skeletal muscle decreased significantly in old (24-mo) WT mice; however, this decrease was remarkably attenuated in the Cisd2 TG mice (FIG. 5G). Of note, the age-associated increases in TFAM and mtDNA copy number, which were detected by us in the WT mice, were found to be absent in the Cisd2 TG mice (FIGS. 5H, 51; FIG. 20F).

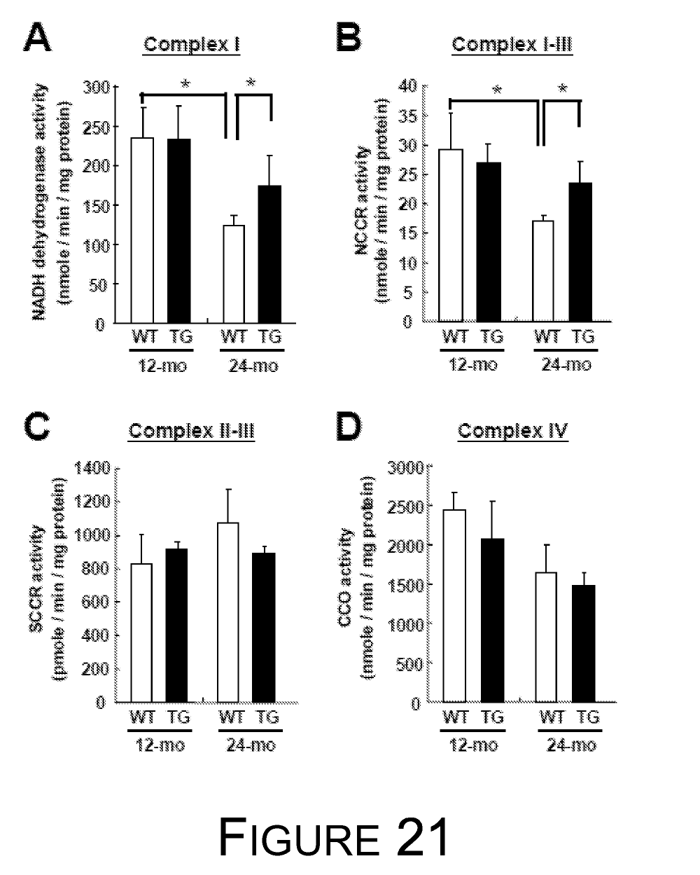

To investigate whether an attenuation of the age-associated damage to mtDNA and an amelioration of reduction in complex proteins have a direct functional benefit, we assessed aerobic respiration using isolated mitochondria prepared from skeletal muscle. Our results revealed an age-dependent decrease in oxygen consumption in both the WT and Cisd2 TG mice; however, there was a remarkable increase in the oxygen consumption of the mitochondria from the Cisd2 TG mice compared to those from the WT controls at the same age (24-mo) (FIG. 5J). To further expand this investigation, we measured the enzyme activities of complex I and IV, and the electron transport activity of complex I-III and complex II-III. Our results revealed a significant increase in the activity levels of complex I and complex I-III in the old (24-mo) Cisd2 TG mice compared to the control mice (FIG. 21); this result is consistent with the observation that Cisd2 TG mice have a reduced level of D-17 deletion, which covers the ND1 and ND2 genes that encode two components of complex I (FIGS. 5D, 5E). Together, these results show that constitutive expression of Cisd2 protects mitochondria and reduces the age-associated decline in aerobic respiration during aging.

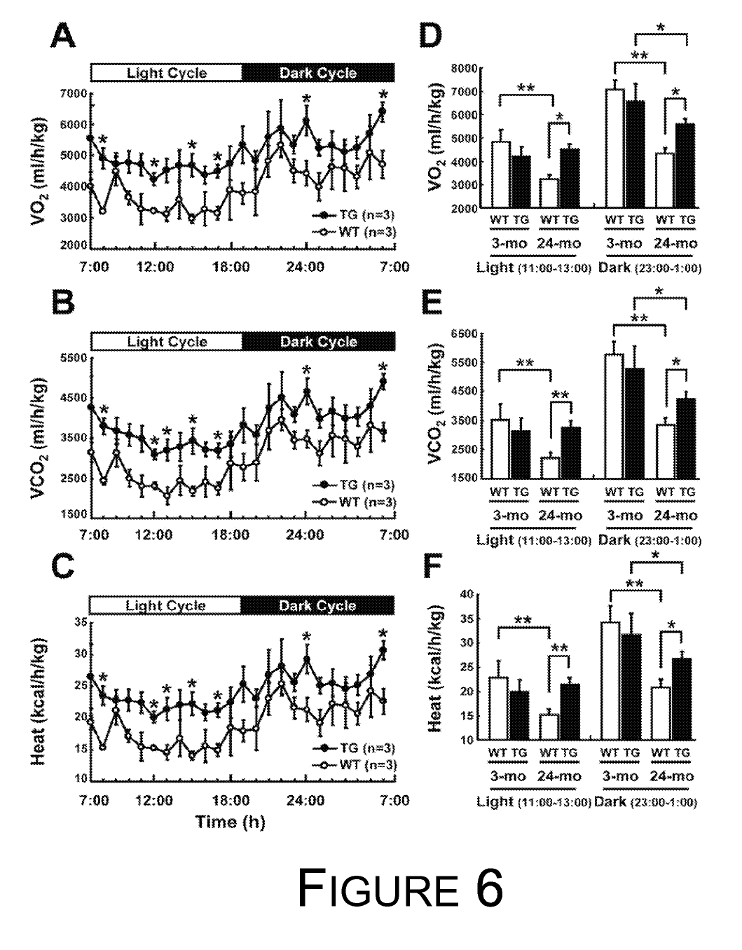

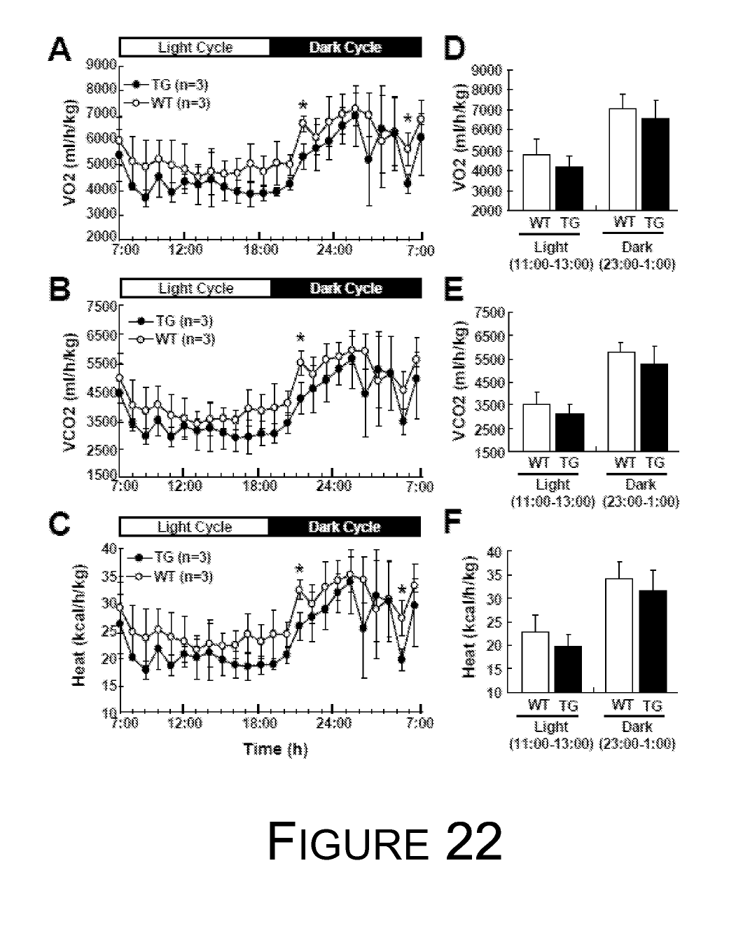

To assess the impact of better preserved mitochondrial function on age-associated changes in whole-body energy metabolism, we monitored the mice by indirect calorimetry. Consistent with the decreased oxygen consumption observed using the isolated mitochondria of old (24-mo) WT mice, whole-body oxygen consumption (VO₂), CO₂ production (VCO₂) and heat generation were significantly decreased in the old (24-mo) WT mice compared to the young (3-mo) WT mice during both light and dark cycles (FIGS. 6A-6F; FIG. 22). In contrast, old (24-mo) Cisd2 TG mice were well protected from these age-associated reductions in O₂ consumption, CO₂ production and heat generation during the light cycle; furthermore, during the dark cycle, although the reduction in these age-associated parameters was not fully protected, the decline was significantly attenuated in the Cisd2 TG mice (FIGS. 6A-6F). Taken together, these results demonstrate that aging is associated with mitochondrial damage and is accompanied by reductions in mitochondrial function and whole-body energy metabolism. Importantly, these deleterious changes can be prevented or significantly ameliorated by a persistent level of Cisd2 expression during aging.

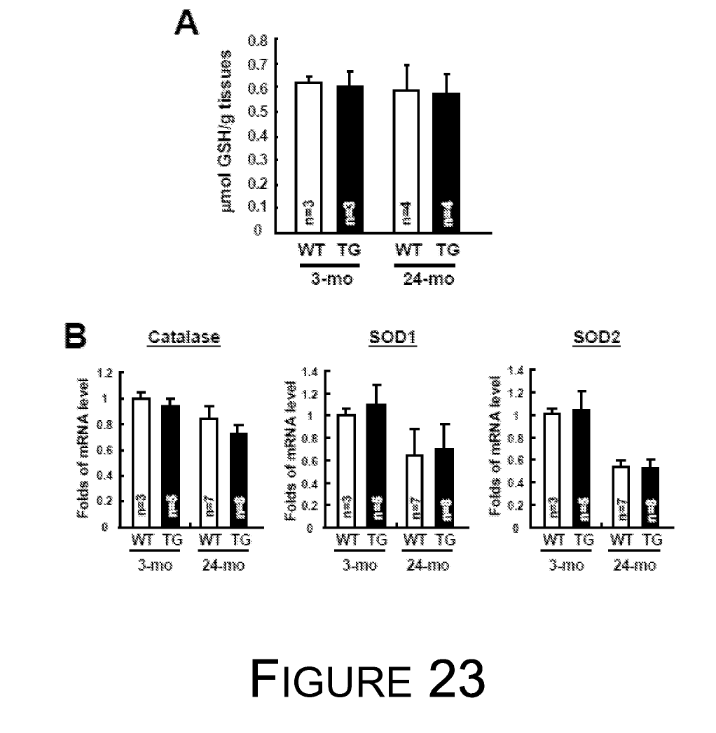

To test whether a decrease in oxidative stress contributes to the longevity phenotype of Cisd2 TG mice, we monitored intracellular glutathione (GSH) levels in the skeletal muscles of young (3-mo) and old (24-mo) WT and Cisd2 TG mice; GSH is an important antioxidant involved in defense against oxidative stress and the maintenance of cellular redox homeostasis. Our result revealed no significant difference in GSH levels at both young and old ages between WT and Cisd2 TG mice (FIG. 23A). In addition, the mRNA levels of the enzymes that scavenge reactive oxygen species (ROS) were also unaffected (FIG. 23B). These findings suggest that modulation of the ROS-induced stress response does not seem to play a significant role in the anti-aging effect of Cisd2 in the TG mice.

Any combination of the above-described elements in all possible variations thereof is encompassed by the present disclosure unless otherwise indicated herein or otherwise clearly contradicted by context.

The terms “a” and “an” and “the” and similar referents as used in the context of de-scribing the present disclosure are to be construed to cover both the singular and the plural, unless otherwise indicated herein or clearly contradicted by context.

The use of any and all examples, or exemplary language (e.g., “such as”) provided herein, is intended merely to better illuminate the present disclosure and does not pose a limitation on the scope of the present disclosure unless otherwise indicated. No language in the specification should be construed as indicating any element is essential to the practice of the present disclosure unless as much is explicitly stated.

The description herein of any aspect or embodiment of the present disclosure using terms such as “comprising”, “having”, “including” or “containing” with reference to an element or elements is intended to provide support for a similar aspect or embodiment of the present disclosure that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The present disclosure includes all modifications and equivalents of the subject matter recited in the aspects or claims presented herein to the maximum extent permitted by applicable law.

1. A therapeutic for treating and/or preventing aging-associated damage, the therapeutic comprising a delivery vehicle carrying a Cisd2 gene.

2. The therapeutic of claim 1, wherein the Cisd2 gene is selected from the group consisting of human Cisd2 gene and murine Cisd2 gene.

3. The therapeutic of claim 1, wherein the delivery vehicle is a vector, a liposome, a polymer, a pharmaceutically acceptable composition, or a device which facilitates delivery of such delivery vehicle.

4. The therapeutic of claim 3, wherein the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

5-6. (canceled)

7. The therapeutic of claim 1, wherein the aging-associated damage is selected from the group consisting of cell injury, tissue damage associated with skin, neuron, muscle, pancreas, brain, kidney, lung, stomach, intestine, spleen, heart, adipose tissue, testes, ovary, uterus, liver or bone, organ dysfunction, aging-associated lifespan shortening and carcinogenesis.

8. (canceled)

9. The therapeutic of claim 7, wherein the skin comprises epidermis, dermis, adipose layers, hair follicles, hair shaft, and sebaceous glands.

10. The therapeutic of claim 1, wherein the delivery vehicle is combined with another unrelated therapy.

11. An expression cassette comprising a polynucleotide comprising a sequence encoding a Cisd2 polypeptide operably linked to a promoter functional in a host cell.

12. The expression cassette of claim 11, wherein the Cisd2 polypeptide is selected from the group consisting of human Cisd2 polypeptide and murine Cisd2 polypeptide.

13. The expression cassette of claim 11, wherein the promoter is a tissue specific promoter.

14. A method of treating and preventing at least one aging-associated condition comprising administering to a mammal in need of such treatment a therapeutically effective amount of a Cisd2 gene.

15. The method of claim 14, wherein the Cisd2 gene is selected from the group consisting of human Cisd2 gene and murine Cisd2 gene.

16. The method of claim 14, wherein the administering step comprises administering using a delivery vehicle.

17. The method of claim 16, wherein the delivery vehicle is a vector, a liposome, a polymer, a pharmaceutically acceptable composition, or a device which facilitates delivery of such delivery vehicle.

18. The method of claim 17, wherein the vector is selected from the group consisting of adenovirus vectors, retrovirus vectors, adeno-associated virus vectors, herpes simplex virus vectors, SV40 vectors, polyoma virus vectors, papilloma virus vectors, picarnovirus vectors, vaccinia virus vectors, lentiviral vectors, alphaviral vectors, a helper-dependent adenovirus, and a plasmid.

19. The method of claim 14, wherein the administering step includes administering in a manner selected from the group consisting of intravenous administration, subcutaneous administration, intra-bone marrow administration, intra-arterial administration, intra-cardiac administration, intracerebral administration, intraspinal administration, intra-peritoneal administration, intra-muscular administration, parenteral administration, intra-rectal administration, intra-tracheal injection, intra-nasal administration, intradermal administration, epidermal administration, oral administration and combinations thereof.

20. The method of claim 14, wherein the aging-associated condition is selected from the group consisting of cell injury, tissue damage associated with skin, neuron, muscle, pancreas, brain, kidney, lung, stomach, intestine, spleen, heart, adipose tissue, testes, ovary, uterus, liver or bone, organ dysfunction, aging-associated lifespan shortening and carcinogenesis.

21. (canceled)

22. The method of claim 20, wherein the skin comprises epidermis, dermis, adipose layers, hair follicles, hair shaft, and sebaceous glands.

23. The method of claim 14, wherein the administering step includes administering to the mammal in need of treatment multiple therapeutically effective amounts of the Cisd2 gene.

24. The method of claim 14, wherein the administering step includes administering the Cisd2 gene in combination with another therapeutic.

25-41. (canceled)


SEQUENCE LISTING (3 sequences)

SEQ ID NO: 1 - DNA, 2885 bp, Mus musculus
ccgcgagcccgccggccgcttccgctctcacgcaagcgcagcagccgggcagcggacgccgccggcggcaggatggtcctggacagcgtggcccgcatcgtgaaggtgcagctgcccgcctacctcaagcagctcccggtccccgacagcatcaccgggttcgcccgcctcacagtttcagactggctccgcctactgcccttcctcggggtacttgcgcttctgggctacctcgcagtgcgcccattcttcccaaagaagaagcaacagaaggatagcttgatcaatcttaagatacaaaaggaaaatcccaaggtggtgaatgagataaacattgaagatctgtgtctcaccaaagcagcttattgtaggtgctggcggtccaagacgtttcctgcctgtgatggatcccataataagcataatgaattgacaggcgataacgtgggtcctctcatcctgaagaagaaagaagtatagcagtggccatttgagatgaaactctgacagagtgtttccatgtgtgttgtgcatagaaaagtctttggaagttttaactctcagtactggttgaataattatttctgccagttattttcttgctacactactgcttatatttggtactttatatagtcagtcatttctatagaaatcagatggtcaagtctcattctgaaattaagtttaatgtgtctttcaacaataaagtcagcacttcagattttaattaattgggaatttaaagtttgactgtgggccaaaagttatttctttgtatatcagtatttcaacagaatacatagttttaaaaaaattatcctttgttctgttaaatgtatctttatattgtcttgttttgctcatttgaagtcatttgccatttttttggccttttgtggaactctgctgcttttagaagaattatgaaaaataattgccatcaacaatattttgaggcagtaaaaccccttgttttatagagactttcatcttattaccatgattatcccaatttctaaaaagtaaatcagatataaaaatttccaattagaaacaattattttcatcataaaaatggttcaccttacaaaagccttgttgtgtgtgtgagggtgtgagggtgtatatgtgtgtgtgagtatgtgtgtgtgtgtgagagagagagagtatgtgtgtgtgtgtgtggtgcttaacttggagactgctgggtggcaagagcactcatgaactagagtttgaagatcagttagatattaaataactgaatttgcattaaaatgatgcatagttcattgctcaaaattatgttttatcttttctgttgtaaattatagagaaacggcaacaggatattaagttcttgtattctaatggattgaatttgtacatactgagagaaaatattttgatcttttttaaaagagataactcttgttgtatttttttaacgtcacaaaatatagatcaaacacttgtgggcataatgacagaaaaaaagtctggaaggaccaaggactatgactatgacacagcatggtccacttgcacggataaggacgaggacttagcaaaggttgctgcttttactctctatttctcttcccccccccccccccccgcaacaaacctttatatgctttgcttttaattgatcttttcttaattgaggctttgaatagaatcgtcatagcacagttaagctctttaggcccgagtgataagacctgattgattgtctaaggatagggtggagccagcagggcaggaagcatcatcacaagtggattagacagaggctgttcgaggagaggaagccacacacgtgttacagcaaatactgaaaaaaccaaaacaccaacagtcctttcacgtctggctcctaagtgaaatgacacgtcaagggcaaggtaggctaaataaagaaggtgggctttaagtatgtacttcagttctgccgtgttcccccagaagcagggtaaaagcaagacagttaaatctggggacactcacctgtctgcctgctggagtagatggaccatctctaggatttcttctttattaaatgagcacacagtatgcctggtaccacatgaattctgtttgttctctttctttagctatcctatctagcagtttgagaaagactagtgatggcaagctaaaaaggaaaatgattaccagtcaacttgtcacaaaattaagttacactgacacagtttgtagtcttagtttgtgtattgtaaagtgtaaataatgttaaaacagacagacatgttttagaaagacttatttcatgtgtatgagtgcctgtctgcctgtctgtgtgtctgtctgtgcaccatgcacatgcctggtgcccgaggaagccagagagaatgttggatccctggaactggaattaacagatagttgtgagctaccatgtgaatactgggaactgaacctggtctcctacaagaacatccagtgctattaacaactgagccatctctctaaccctataaaacttttaataaaagaggttatgattggtttgaagtagcaactgaaatgtggaaggaagtattccgaaaggcagaagtggtacaaagccgtgtctgctcactccactcatggtactgaggatggaacccagggcctcccacaccctggcaagagcccttcacactgagctccgtcttgagttgtatcttttcaatagcataaaccattgagacaaaatacaagaaagttttccatataggtttactggaatttttgaattatttgtgattttagcctactatcagaagtctctttagttttattttacaagtctcatgtaagagctattcttaaactattaaagatacaacctactcgttcaaaaaaaaaaaaaaaaa

SEQ ID NO: 2 - DNA, 26 bp, Artificial
  primer
gccagcctgacccatagccataatat

SEQ ID NO: 3 - DNA, 24 bp, Artificial
  primer
attaataaggccaggaccaaacct